(19) United States
(12) Patent Application Publication
(10) Pub. No.: US 2025/0283041 A1
(43) Pub. Date: Sep. 11, 2025

(54) METHODS FOR PRODUCING RED BLOOD CELLS

(71) Applicant: Albert Einstein College of Medicine, Bronx, NY (US)

(72) Inventors: Eric Bouhassira, Hastings On Hudson, NY (US); Emmanuel Olivier, Pelham, NY (US); Zi Yan, Bronx, NY (US)

(21) Appl. No.: 18/862,575

(22) PCT Filed: May 4, 2023
(86) PCT No.: PCT/US2023/066608

§ 371 Date: Nov. 4, 2024

Related U.S. Application Data
(60) Provisional application No. 63/345,793, filed on May 25, 2022.

(51) Int. Cl. C12N 5/078; C07K 14/715

(52) U.S. Cl. CPC C12N 5/0641; C07K 14/715; C12N 2500/25; C12N 2501/115; C12N 2501/14; C12N 2501/145; C12N 2501/155; C12N 2501/165; C12N 2501/39; C12N 2501/73; C12N 2501/91; C12N 2506/45; C12N 2510/00

(57) ABSTRACT

This disclosure is based, at least in part, on the unexpected discovery that genetically modified pluripotent stem cells having a constitutively active stem cell factor (SCF) can differentiate into enucleated red blood cells, self-renew, and expand in culture media for an extended period of time. Accordingly, the disclosed methods enable large-scale production of red blood cells with significantly reduced overall cost as compared to the existing methods.

### CROSS-REFERENCE TO RELATED APPLICATIONS

This application claims priority under 35 U.S.C. § 119 (e) to U.S. Provisional Patent Application No. 63/345,793, filed May 25, 2022. The foregoing application is incorporated by reference herein in its entirety.

### STATEMENT REGARDING FEDERALLY SPONSORED RESEARCH

This invention was made with government support under 2R01HL130764-07 awarded by the National Institutes of Health. The government has certain rights in the invention.

### FIELD OF THE INVENTION

The present invention relates generally to methods for producing red blood cells by differentiating genetically modified pluripotent stem cells.

### BACKGROUND OF THE INVENTION

Blood transfusions have been clinically useful for more than 100 years, and the idea that red blood cells (RBCs) could be modified to serve as more than just oxygen carriers is almost as old. Drug delivery through therapeutic red blood cells that spatially restricts the drugs to the lumen of the cardio-vascular system and shields the drug from the immune system results in increased half life and reduced toxicity and risks of allergy.

As cell culture methods and genetic engineering methods have progressed, in vitro production of red blood cells has become an alternate strategy to generate red blood cells for transfusion and to load red blood cells with drugs. One major advantage of in vitro production is that genetically homogeneous cells can be produced from the stem cells of a single rare donor carrying desirable blood groups that are compatible with a very large fraction of the population. If the source cells are immortal, an unlimited number of cells can be produced, which eliminates the risk of contamination by unknown or emerging pathogens associated with collection of cells from donors and decreases production complications associated with the genetic heterogeneity of the donors.

Existing methods to differentiate adult or cord blood primary hematopoietic stem and progenitor cells (HSPCs) into red blood cells in culture could be useful to treat a few patients or to generate small batches of therapeutic red blood cells. However, HSPCs expansion does not eliminate the need to collect cells from volunteers. Immortalization protocols that yield cell lines that can differentiate into red blood cells and enucleate at modest rates have been developed. However, all lines produced so far are karyotypically unstable, exhibit a low growth rate, and can only be cultured at low density.

Therefore, there remains a strong need for developing improved methods for large-scale production of red blood cells.

### SUMMARY OF THE INVENTION

This disclosure addresses the need mentioned above in a number of aspects. In one aspect, this disclosure provides a method of producing red blood cells.

In some embodiments, the method comprises: (a) providing a genetically modified pluripotent stem cell comprising a mutant cytokine receptor that is constitutively active; and (b) differentiating the genetically modified pluripotent stem cell into the red blood cell by culturing the genetically modified pluripotent stem cell or a derivative thereof in one or more culture media free of a cytokine.

In some embodiments, the method comprises: (a) providing a genetically modified pluripotent stem cell comprising a constitutively active stem cell factor (SCF) receptor; and (b) differentiating the genetically modified pluripotent stem cell into a red blood cell by culturing the pluripotent stem cell or a derivative thereof in one or more culture media free of a stem cell factor (SCF).

In some embodiments, the SCF receptor (also referred to as the kit gene) comprises a D816V mutation. In some embodiments, the SCF receptor comprises the amino acid sequence of SEQ ID NO: 2.

In some embodiments, the method comprises culturing the genetically modified pluripotent stem cell or a derivative thereof in a medium comprising erythropoietin, dexamethasone, and isobutylmethylxanthine, 1-Methyl-3-Iso-butyl-xanthine (IBMX).

In some embodiments, the medium comprises from about 0.1 to about 10 units of erythropoietin. In some embodiments, the medium comprises from about 1 unit of erythropoietin.

In some embodiments, the medium comprises from about 0.01 μM to about 100 μM of dexamethasone. In some embodiments, the medium comprises about 1 μM of dexamethasone.

In some embodiments, the medium comprises from about 1 μM to 500 M of IBMX. In some embodiments, the medium comprises about 33 μM of IBMX. In some embodiments, the medium comprises about 1 unit of erythropoietin, about 1 μM dexamethasone, and about 33 μM of IBMX.

In some embodiments, the method comprises removing the dexamethasone and the IBMX from the medium to induce differentiation of the genetically modified pluripotent stem cell into an enucleated cell.

In some embodiments, the method comprises refreshing the medium at least once every first interval by adding a concentrated medium comprising erythropoietin, dexamethasone, and IBMX, and passaging cells by dilution at least once every second interval, while maintaining the concentration of the cells between 200,000 and 2,000,000 cells.

In some embodiments, the first interval is about 2 days. In some embodiments, the second interval is about 7 days.

In some embodiments, the one or more culture media are free of the stem cell factor and erythropoietin.

In some embodiments, the one or more culture media comprise a serum-free medium. In some embodiments, the one or more culture media comprise a defined differentiation medium. In some embodiments, the culture medium comprises one or more of vascular endothelial growth factor (VEGF), bone morphogenic protein (BMP), Flt-3L (FL), thrombopoietin (TPO), interleukin-3 (IL-3), interleukin-6 (IL-6), and heparin.

In some embodiments, the one or more culture media comprise a supplement selected from inositol, folic acid, monothioglycerol, transferrin, insulin, ferrous nitrate, ferrous sulfate, BSA, L-glutamine, penicillin-streptomycin, and combinations thereof.

In some embodiments, the method comprises supplementing the culture medium with a cytokine only from day 0 to day 17. In some embodiments, the method further comprises expanding the pluripotent stem cell prior to being differentiated into the red blood cell.

In some embodiments, the pluripotent stem cell is cultured using a bioreactor. In some embodiments, the method further comprises differentiating the pluripotent stem cell into a progenitor cell and sorting a population of progenitor cells differentiated from the pluripotent stem cell using magnetic-activated cell sorting (MACS), flow cytometry, or fluorescence-activated cell sorting (FACS). In some embodiments, the method further comprises sorting the population of progenitor cells based on the expression of one or more of CD31, CD34, CD43, and CD45.

In some embodiments, the method further comprises dispersing a population of pluripotent stem cells by treatment with one or more enzymes. In some embodiments, the one or more enzymes comprise trypsin or TrypLE.

In some embodiments, the red blood cell expresses ADAMTS13, asparaginase, Factor VIII, Factor IX, or phenylalanine hydroxylase.

In some embodiments, the pluripotent stem cell comprises an embryonic stem cell or an embryo-derived cell. In some embodiments, the pluripotent stem cell comprises an induced pluripotent stem cell (iPSC). In some embodiments, the pluripotent stem cell is a human cell.

In another aspect, this disclosure also provides blood, cellular and acellular blood components, or blood products obtained from the red blood cell produced by the method disclosed herein.

Also within the scope of this disclosure is a composition comprising the red blood cell produced by the method described herein, or the blood, cellular and acellular blood components, or blood products described herein. In some embodiments, the composition comprises a cryo-protectant.

In yet another aspect, this disclosure provides a kit comprising the red blood cell produced by the method described herein, the blood, cellular and acellular blood components, or blood products, or the composition, as described herein. In some embodiments, the kit further comprises a defined culture medium.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIGS. 1A, 1B, 1C, ID, and 1E are a set of diagrams showing production of red blood cells using the Pluripotent Stem Cell-Robust Erythroid Differentiation (PSC-RED) protocol. FIG. 1A shows the yield of PSC-RED as compared to an existing commonly used protocol. One iPSC yields more than 100,000 erythroid cells. FIG. 1B shows the results of FACS analysis demonstrating about 50% enucleation. Draq5: nuclear stain. FIG. 1C shows the results of Giemsa staining after filtration of nuclei and orthochromatic erythroblasts, demonstrating production of a 99% pure batch of enucleated RBCs from iPSCs. FIG. 1D shows that the results of Globin analysis by HPLC of enucleated RBCs demonstrated expression of about 15% adult chains and 80% fetal chains. FIG. 1E shows that as all RBCs produced in culture, iPSC-derived RBCs are slightly larger than adult RBCs produced in vivo, indicating they are primarily reticulocytes.

FIG. 2 shows a chromatogram illustrating introduction of the D816V mutation in iPSC line O1. Hemizygous line has the D816V mutation on one allele and a single nucleotide deletion on the allele (control sequence: SEQ ID NO: 6; homozygous D816V: SEQ ID NO: 7; and hemizygous D816V: SEQ ID NO: 8).

FIGS. 3A and 3B are a set of diagrams showing the effect of the D816V kit mutation. FIG. 3A shows that the PSC-RED protocol requires SCF from day 2 to 38 and Epo from day 17 to 38. The only cytokines needed after day 17 are SCF and Epo. FIG. 3B shows expansion of control iPSC (grown with SCF and Epo) compared to D816V hemizygous and homozygous clones grown without SCF and EPO.

FIGS. 4A and 4B are a set of diagrams showing the results of FACS analysis of differentiating iPSCs with or without the D816V mutation. The analysis on a Cytek Aurora with 15 markers revealed a complex pattern of differentiation in the three iPSC sub-lines. FIG. 4A shows that the evolution of the cells in culture can be recapitulated by dividing the cells into 8 major populations defined by expression of CD43, CD34, CD31, CD45, CD235a, CD71, and CD117. FIG. 4B shows that at day 10, the phenotypes of the cultures of the unmodified and of the two kit mutated lines were similar and composed mostly of populations of CD43+CD45-CD34+ HPSCs which differed by expression of CD235a.

FIGS. 5A, 5B, 5C, 5D, and 5E are a set of diagrams showing enucleation and globin gene expression. FIG. 5A is a graph illustrating acceleration of differentiation induced by dasatinib. FIG. 5B is a dot plot illustrating the rate of enucleation of line A4 at day 41 with or without 200 nM dasatinib. FIG. 5C is a micrograph illustrating the morphology of the orthochromatic erythroblasts and of the enucleated cells obtained from clone A4 at day 41. FIGS. 5D and 5E show chromatograms and bar graphs illustrating globin expression observed at day 41 in erythroblasts collected from control cells and from clones A4 and B34.

FIGS. 6A, 6B, 6C, and 6D show an alternative protocol to generate and differentiate late CFU-E/pro Erythroblast-like self-renewing Kit-A4-DEI cells. FIG. 6A shows that iPSCs were differentiated until day 17 and then placed in a culture medium termed IMIT-DEI composed of IMIT plus 1 unit of erythropoietin/mL, 33 μM IBMX, and 1 μM dexamethasone and expanded for long periods of times (up to 135 days). During this expansion period cells were periodically differentiated into enucleated erythroid cells by culturing for 5 to 9 days (generally 7 days) in a medium composed of IMIT and 4 unit of erythropoietin for 7 days yielding a mixture of mature erythroid cells including more than 20% enucleated cells. DEI: Dexametasona, Erythropoiein (Epo) IBMX. E: erythropoietin. FIG. 6B is a graph illustrating the expansion of the Kit-A4-DEI cells. Cell number increased an average of 8.5-fold per week. FIG. 6C (Left) shows a flow cytometry profile of the kit-A4-DEI. Kit-A4-DEI cells were tested weekly using a panel of antibodies and exhibited a fairly constant antigen profile. Kit-A4-DEI cells are >98% positive for CD36, CD43, and CD71; a majority of the cells also express CD117 and CD235a; a minority of cells express CD34 and CD45 and almost all cells are negative for CD45RA. When grown in IMIT-DEI the kit-A4-DEI cells can self-renew and have a phenotype similar to late CFU-E/proerythroblasts. FIG. 6C (Right) is a micrograph illustrating the morphology of kit-A4-DEI cells maintained in IMIT-DEI after Romanovsky staining. FIG. 6D shows a dotplot and micrograph illustrating an experiment in which kit-A4-DEI cells (previously expanded for 125 days in IMIT-DEI) were differentiated for 7 days in an erythroid maturation medium consisting of IMIT+4 unit of erythropoietin. During the 7-day culture the cells expanded an average of 4 to 5 times and differentiated into a mixture of mature erythroid cells including more than 20% enucleated cells (average 22.3% n=3) as determined by flow cytometry or microscopy after Romanovsky staining.

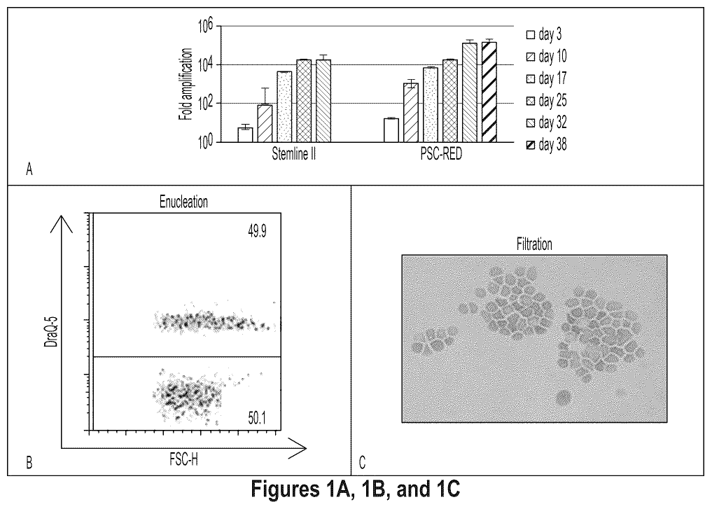

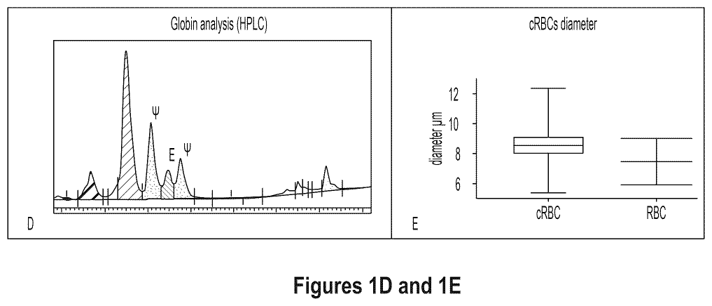

### DETAILED DESCRIPTION OF THE INVENTION

This disclosure is based, at least in part, on the unexpected discovery that genetically modified pluripotent stem cells having a constitutively active stem cell factor (SCF) can differentiate into enucleated red blood cells, self-renew, and expand in culture media for an extended period of time. Accordingly, the disclosed methods enable large-scale production of red blood cells with significantly reduced overall cost (e.g., at least 10-20 fold) as compared to the existing methods.

### Methods of Producing Red Blood Cells

In one aspect, this disclosure provides a novel, improved method of producing red blood cells. In some embodiments, the method comprises: (a) providing a genetically modified pluripotent stem cell comprising a constitutively active SCF receptor; and (b) differentiating the genetically modified pluripotent stem cell into a red blood cell by culturing the pluripotent stem cell or a derivative thereof in one or more culture media free or substantially free of a stem cell factor (SCF).

As used herein, the term “differentiate” refers to the production of a cell type that is more differentiated than the cell type from which it is derived. In some embodiments, the term “differentiate” means to produce a cell that has fewer fate choices than the cell from which it was derived. The term, therefore, encompasses cell types that are partially and terminally differentiated. Differentiated cells derived from pluripotent stem cells are generally referred to as pluripotent stem cell-derived cells or pluripotent stem cell-derived cell aggregate cultures, pluripotent stem cell-derived single cell suspensions, pluripotent stem cell-derived cell adherent cultures, or the like.

In some embodiments, the method comprises culturing the genetically modified pluripotent stem cell or a derivative thereof in a medium comprising erythropoietin, dexamethasone, and/or isobutylmethylxanthine, 1-Methyl-3-Iso-butyl-xanthine (IBMX).

In some embodiments, the method comprises culturing the genetically modified pluripotent stem cell or a derivative thereof in a medium comprising erythropoietin, dexamethasone, and isobutylmethylxanthine, 1-Methyl-3-Iso-butyl-xanthine (IBMX). In some embodiments, a derivative of a genetically modified pluripotent stem cell can be resulted from culturing or expansion in a medium different from the medium comprising erythropoietin, dexamethasone, and isobutylmethylxanthine, 1-Methyl-3-Iso-butyl-xanthine (IBMX). In some embodiments, a derivative of a genetically modified pluripotent stem cell can be a product of division or differentiation of the genetically modified pluripotent stem cell.

In some embodiments, the medium comprises from about 0.1 to about 10 units (e.g., 0.1, 0.3, 0.5, 0.7, 0.9, 1.1, 1.3, 1.5, 1.7, 1.9, 2.1, 2.3, 2.5, 2.7, 2.9, 3.1, 3.3, 3.5, 3.7, 3.9, 4.1, 4.3, 4.5, 4.7, 4.9, 5.1, 5.3, 5.5, 5.7, 5.9, 6.1, 6.3, 6.5, 6.7, 6.9, 7.1, 7.3, 7.5, 7.7, 7.9, 8.1, 8.3, 8.5, 8.7, 8.9, 9.1, 9.3, 9.5, 9.7, 9.9, 10 units) of erythropoietin. In some embodiments, the medium comprises from about 1 unit of erythropoietin.

In some embodiments, the medium comprises from about 0.01 μM to about 100 μM (e.g., 0.01, 0.1, 0.5, 1, 5, 10, 15, 20, 25, 30, 35, 40, 45, 50, 55, 60, 65, 70, 75, 80, 85, 90, 95, 100 μM) of dexamethasone. In some embodiments, the medium comprises about 1 μM of dexamethasone.

In some embodiments, the medium comprises from about 1 μM to 500 μM (e.g., 1, 5, 10, 15, 20, 25, 30, 33, 35, 40, 45, 50, 55, 60, 65, 70, 75, 80, 85, 90, 95, 100 μM) of IBMX. In some embodiments, the medium comprises about 33 μM of IBMX. In some embodiments, the medium comprises about 1 unit of erythropoietin, about 1 μM dexamethasone, and about 33 μM of IBMX.

In some embodiments, the method comprises removing the dexamethasone and the IBMX from the medium to induce differentiation of the genetically modified pluripotent stem cell into an enucleated cell.

In some embodiments, the method comprises refreshing the medium at least once every first interval by adding a concentrated medium comprising erythropoietin, dexamethasone, and IBMX, and passaging cells by dilution at least once every second interval, while maintaining the concentration of the cells between 200,000 and 2,000,000 cells.

In some embodiments, the first interval is from about 1 day to about 7 days (e.g., 1, 2, 3, 4, 5, 6, 7 days). In some embodiments, the first interval is about 2 days.

In some embodiments, the second interval is from about 1 day to about 30 days (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days). In some embodiments, the second interval is about 7 days.

In some embodiments, one or more culture media free or substantially free of a stem cell factor (SCF) and/or erythropoietin (Epo).

As used herein, the term “substantially free,” such as “substantially free of Epo,” “substantially free of SCF,” or “substantially free of serum,” is meant that the solution, media, supplement, excipient, and the like, is at least 98%, or at least 98.5%, or at least 99%, or at least 99.5%, or at least 100% free of Epo, SCF, or serum. In some embodiments, a defined culture media contains no SCF or is 100% SCF-free, or is substantially free of SCF. In some embodiments, a defined culture media contains no Epo or is 100% Epo-free, or is substantially free of Epo. In some embodiments, a defined culture media contains no Epo and SCF or is 100% Epo-free and SCF-free, or is substantially free of Epo and SCF.

In some embodiments, the method comprises differentiating the pluripotent stem cell into enucleated erythroid cells (such as reticulocyte and red blood cells). “Erythroid cells,” as used herein, include nucleated red blood cells, red blood cell precursors, and enucleated red blood cells. As used herein, the term “enucleated” refers to a cell, e.g., a reticulocyte or mature red blood cell (erythrocyte), that lacks a nucleus. In some embodiments, the method comprises differentiating the pluripotent stem cell into nucleated red erythroid precursor and progenitor cells.

As used herein, “constitutively active SCF receptor” refers to a SCF receptor capable of performing all or some of the functions of the unmodified SCF receptor in a ligand-independent manner, for example, without contacting the SCF or Epo molecule or any other natural ligand. In some embodiments, the unmodified SCF receptor comprises the amino acid sequence of SEQ ID NO: 1. In some embodiments, a constitutively active SCF receptor comprises the amino acid sequence of SEQ ID NO: 2.

In some embodiments, the SCF receptor (also referred to as kit) comprises one or more mutations that render the SCF receptor constitutively active. In some embodiments, the SCF receptor comprises a D816V mutation, rendering the SCF receptor constitutively active. Because the SCF receptor hetero-dimerizes with the Epo receptor, the D816V mutation eliminates the need for both Epo and SCF.

SCF receptor
(SEQ ID NO: 1)
RGARGAWDFLCVLLLLLRVQTGSSQPSVSPGEPSPPSIHPGKSDLIVRVG
DEIRLLCTDPGFVKWTFEILDETNENKQNEWITEKAEATNTGKYTCTNKH
GLSNSIYVFVRDPAKLFLVDRSLYGKEDNDTLVRCPLTDPEVTNYSLKGC
QGKPLPKDLRFIPDPKAGIMIKSVKRAYHRLCLHCSVDQEGKSVLSEKFI
LKVRPAFKAVPVVSVSKASYLLREGEEFTVTCTIKDVSSSVYSTWKRENS
QQTKLQEKYNSWHHGDFNYERQATLTISSARVNDSGVFMCYANNTFGSAN
VTTTLEVVDKGFINIFPMINTTVFVNDGENVDLIVEYEAFPKPEHQQWIY
MNRTFTDKWEDYPKSENESNIRYVSELHLTRLKGTEGGTYTFLVSNSDVN
AAIAFNVYVNTKPEILTYDRLVNGMLQCVAAGFPEPTIDWYFCPGTEQRC
SASVLPVDVQTLNSSGPPFGKLVVQSSIDSSAFKHNGTVECKAYNDVGKT
SAYFNFAFKGNNKEQIHPHTLFTPLLIGFVIVAGMMCIIVMILTYKYLQK
PMYEVQWKVVEEINGNNYVYIDPTQLPYDHKWEFPRNRLSFGKTLGAGAF
GKVVEATAYGLIKSDAAMTVAVKMLKPSAHLTEREALMSELKVLSYLGNH
MNIVNLLGACTIGGPTLVITEYCCYGDLLNFLRRKRDSFICSKQEDHAEA
ALYKNLLHSKESSCSDSTNEYMDMKPGVSYVVPTKADKRRSVRIGSYIER
DVTPAIMEDDELALDLEDLLSFSYQVAKGMAFLASKNCIHRDLAARNILL
THGRITKICDFGLARDIKNDSNYVVKGNARLPVKWMAPESIFNCVYTFES
DVWSYGIFLWELFSLGSSPYPGMPVDSKFYKMIKEGFRMLSPEHAPAEMY
DIMKTCWDADPLKRPTFKQIVQLIEKQISESTNHIYSNLANCSPNRQKPV
VDHSVRINSVGSTASSSQPLLVHDDV
SCF receptor with D816V mutation
(SEQ ID NO: 2)
RGARGAWDFLCVLLLLLRVQTGSSQPSVSPGEPSPPSIHPGKSDLIVRVG
DEIRLLCTDPGFVKWTFEILDETNENKQNEWITEKAEATNTGKYTCTNKH
GLSNSIYVFVRDPAKLFLVDRSLYGKEDNDTLVRCPLTDPEVTNYSLKGC
QGKPLPKDLRFIPDPKAGIMIKSVKRAYHRLCLHCSVDQEGKSVLSEKFI
LKVRPAFKAVPVVSVSKASYLLREGEEFTVTCTIKDVSSSVYSTWKRENS
QQTKLQEKYNSWHHGDFNYERQATLTISSARVNDSGVFMCYANNTFGSAN
VTTTLEVVDKGFINIFPMINTTVFVNDGENVDLIVEYEAFPKPEHQQWIY
MNRTFTDKWEDYPKSENESNIRYVSELHLTRLKGTEGGTYTFLVSNSDVN
AAIAFNVYVNTKPEILTYDRLVNGMLQCVAAGFPEPTIDWYFCPGTEQRC
SASVLPVDVQTLNSSGPPFGKLVVQSSIDSSAFKHNGTVECKAYNDVGKT
SAYFNFAFKGNNKEQIHPHTLFTPLLIGFVIVAGMMCIIVMILTYKYLQK
PMYEVQWKVVEEINGNNYVYIDPTQLPYDHKWEFPRNRLSFGKTLGAGAF
GKVVEATAYGLIKSDAAMTVAVKMLKPSAHLTEREALMSELKVLSYLGNH
MNIVNLLGACTIGGPTLVITEYCCYGDLLNFLRRKRDSFICSKQEDHAEA
ALYKNLLHSKESSCSDSTNEYMDMKPGVSYVVPTKADKRRSVRIGSYIER
DVTPAIMEDDELALDLEDLLSFSYQVAKGMAFLASKNCIHRDLAARNILL
THGRITKICDFGLARVIKNDSNYVVKGNARLPVKWMAPESIFNCVYTFES
DVWSYGIFLWELFSLGSSPYPGMPVDSKFYKMIKEGFRMLSPEHAPAEMY
DIMKTCWDADPLKRPTFKQIVQLIEKQISESTNHIYSNLANCSPNRQKPV
VDHSVRINSVGSTASSSQPLLVHDDV

In some embodiments, the SCF receptor comprises the amino acid sequence of SEQ ID NO: 2.

In some embodiments, the SCF receptor comprises a substitution of D816V or a conservative substitution of Val at residue D816 of the SCF receptor. Examples of conservative substitutions of Val may include Ile, Leu, Met, Phe, and Ala.

Also within the scope of this disclosure are the variants and homologs with significant identity to the SCF receptor. For example, such variants and homologs may have sequences with at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the sequences of the SCF receptor described herein.

As used herein, the term “variant” refers to a first composition (e.g., a first molecule) that is related to a second composition (e.g., a second molecule, also termed a “parent” molecule). The variant molecule can be derived from, isolated from, based on or homologous to the parent molecule. The term variant can be used to describe either polynucleotides or polypeptides.

As applied to polynucleotides, a variant molecule can have an entire nucleotide sequence identity with the original parent molecule, or alternatively, can have less than 100% nucleotide sequence identity with the parent molecule. For example, a variant of a gene nucleotide sequence can be a second nucleotide sequence that is at least 50%, 60%, 70%, 80%, 90%, 95%, 98%, 99% or more identical in nucleotide sequence compared to the original nucleotide sequence. Polynucleotide variants also include polynucleotides comprising the entire parent polynucleotide, and further comprising additional fused nucleotide sequences. Polynucleotide variants also include polynucleotides that are portions or subsequences of the parent polynucleotide; for example, unique subsequences (e.g., as determined by standard sequence comparison and alignment techniques) of the polynucleotides disclosed herein are also encompassed by this disclosure.

In another aspect, polynucleotide variants include nucleotide sequences that contain minor, trivial or inconsequential changes to the parent nucleotide sequence. For example, minor, trivial or inconsequential changes include changes to nucleotide sequence that (i) do not change the amino acid sequence of the corresponding polypeptide, (ii) occur outside the protein-coding open reading frame of a polynucleotide, (iii) result in deletions or insertions that may impact the corresponding amino acid sequence, but have little or no impact on the biological activity of the polypeptide, or (iv) result in the substitution of an amino acid with a chemically similar amino acid. In the case where a polynucleotide does not encode for a protein (for example, a tRNA or a crRNA or a tracrRNA), variants of that polynucleotide can include nucleotide changes that do not result in loss of function of the polynucleotide. In another aspect, conservative variants of the disclosed nucleotide sequences that yield functionally identical nucleotide sequences are encompassed by the invention. One of skill in the art will appreciate that many variants of the disclosed nucleotide sequences are encompassed by this disclosure.

As applied to proteins, a variant polypeptide can have an entire amino acid sequence identity with the original parent polypeptide, or alternatively, can have less than 100% amino acid identity with the parent protein. For example, a variant of an amino acid sequence can be a second amino acid sequence that is at least 50%, 60%, 70%, 80%, 90%, 95%, 98%, 99% or more identical in amino acid sequence compared to the original amino acid sequence.

Polypeptide variants include polypeptides comprising the entire parent polypeptide, and further comprising additional fused amino acid sequences. Polypeptide variants also include polypeptides that are portions or subsequences of the parent polypeptide; for example, unique subsequences (e.g., as determined by standard sequence comparison and alignment techniques) of the polypeptides disclosed herein are also encompassed by the invention.

A “functional variant” of a protein as used herein refers to a variant of such protein that retains at least partially the activity of that protein. Functional variants may include mutants (which may be insertion, deletion, or replacement mutants), including polymorphs, etc. Also included within functional variants are fusion products of such protein with another, usually unrelated, nucleic acid, protein, polypeptide or peptide. Functional variants may be naturally occurring or may be man-made.

In some embodiments, a variant of the SCF receptor may include one or more conservative modifications. The variant of the SCF receptor variant with one or more conservative modifications may retain the desired functional properties, which can be tested using the functional assays known in the art.

As used herein, the term “conservative sequence modifications” refers to amino acid modifications that do not significantly affect or alter the binding characteristics of the protein containing the amino acid sequence. Such conservative modifications include amino acid substitutions, additions, and deletions. Modifications can be introduced by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include: amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, tryptophan); nonpolar side chains (e.g., alanine, valine, leucine, isoleucine, proline, phenylalanine, methionine); beta-branched side chains (e.g., threonine, valine, isoleucine); and aromatic side chains (e.g., tyrosine, phenylalanine, tryptophan, histidine). The SCF receptor with one or more conservative modifications may retain the desired functional properties, which can be tested using the functional assays known in the art.

The percent identity between two amino acid sequences can be determined using the algorithm of E. Meyers and W. Miller (Comput. Appl. Biosci., 4:11-17 (1988)), which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm, which has been incorporated into the GAP program in the GCG software package (available at www.gcg.com), using either a Blossum62 matrix or a PAM250 matrix, and a gap weight of 16, 14, 12, 10, 8, 6, or 4 and a length weight of 1, 2, 3, 4, 5, or 6.

Additionally or alternatively, the protein sequences of this disclosure can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al. (1997) Nucleic Acids Res. 25 (17): 3389-3402. When utilizing BLAST and Gapped BLAST programs, the default parameters of the respective programs (e.g., XBLAST and NBLAST) can be used. (See www.ncbi.nlm.nih.gov).

In some embodiments, the method comprises genetically modifying the pluripotent stem cell prior to differentiation. In some embodiments, the genetic modification comprises introducing a D816V substitution into the SCF receptor.

In some embodiments, genome-editing techniques, such as CRISPR/Cas9 systems, designer zinc fingers, transcription activator-like effectors (TALEs), or homing meganucleases, are available to carry out the genetic modification. In general, “CRISPR/Cas9 system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. One or more elements of a CRISPR system may be derived from a type I, type II, or type III CRISPR system. Alternatively, one or more elements of a CRISPR system may be derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system).

In addition, a wide variety of vectors can be used for the genetic modification as described. The ability of certain viruses to infect cells or enter cells via receptor-mediated endocytosis, and to integrate into host cell genome and express viral genes stably and efficiently have made them attractive candidates for the transfer of foreign nucleic acids into cells. Accordingly, in some embodiments, a viral vector is used to introduce a nucleotide sequence encoding a SCF receptor or fragment thereof. The viral vector will comprise a nucleotide sequence encoding a SCF receptor or fragment thereof operably linked to one or more control sequences, for example, a promoter. Alternatively, the viral vector may not contain a control sequence and will instead rely on a control sequence within the host cell to drive expression of the SCF receptor or fragment thereof. Non-limiting examples of viral vectors that may be used to deliver a nucleic acid include adenoviral vectors, AAV vectors, and retroviral vectors.

In some embodiments, an adeno-associated virus (AAV) can be used to introduce a nucleotide sequence encoding an ADAMTS13 protein or fragment thereof into a host cell for expression. AAV systems have been described previously and are generally well known in the art (Kelleher and Vos, Biotechniques, 17 (6): 1110-7, 1994; Cotten et al., Proc Natl Acad Sci USA, 89 (13): 6094-6098, 1992; Curiel, Nat Immun, 13 (2-3): 141-64, 1994; Muzyczka, Curr Top Microbiol Immunol, 158:97-129, 1992). Details concerning the generation and use of rAAV vectors are described, for example, in U.S. Pat. Nos. 5,139,941 and 4,797,368, each incorporated herein by reference in its entirety for all purposes.

In some embodiments, a retroviral expression vector can be used to introduce a nucleotide sequence encoding a SCF receptor or fragment thereof into a host cell for expression. These systems have been described previously and are generally well known in the art (Nicolas and Rubinstein, In: Vectors: A survey of molecular cloning vectors and their uses, Rodriguez and Denhardt, eds., Stoneham: Butterworth, pp. 494-513, 1988; Temin, In: Gene Transfer, Kucherlapati (ed.), New York: Plenum Press, pp. 149-188, 1986).

Examples of vectors for eukaryotic expression in mammalian cells include AD5, pSVL, pCMV, pRc/RSV, pcDNA3, pBPV, etc., and vectors derived from viral systems such as vaccinia virus, adeno-associated viruses, herpes viruses, retroviruses, etc., using promoters such as CMV, SV40, EF-1, UbC, RSV, ADV, BPV, and β-actin.

Combinations of retroviruses and an appropriate packaging line may also find use, where the capsid proteins will be functional for infecting the target cells. Usually, the cells and virus will be incubated for at least about 24 hours in the culture medium. The cells are then allowed to grow in the culture medium for short intervals in some applications, e.g., 24-73 hours, or for at least two weeks, and may be allowed to grow for five weeks or more, before analysis. Commonly used retroviral vectors are “defective,” i.e., unable to produce viral proteins required for productive infection. Replication of the vector requires growth in the packaging cell line. The host cell specificity of the retrovirus is determined by the envelope protein env (pl20). The envelope protein is provided by the packaging cell line. Envelope proteins are of at least three types, ecotropic, amphotropic, and xenotropic. Retroviruses packaged with ecotropic envelope protein, e.g., MMLV, are capable of infecting most murine and rat cell types. Ecotropic packaging cell lines include BOSC23. Retroviruses bearing amphotropic envelope protein, e.g., 4070A, are capable of infecting most mammalian cell types, including human, dog, and mouse. Amphotropic packaging cell lines include PA12 and PA317. Retroviruses packaged with xenotropic envelope protein, e.g., AKR env, are capable of infecting most mammalian cell types, except murine cells. The vectors may include genes that must later be removed, e.g., using a recombinase system such as Cre/Lox, or the cells that express them destroyed, e.g., by including genes that allow selective toxicity such as herpesvirus TK, bcl-xs, etc. Suitable inducible promoters are activated in a desired target cell type, either the transfected cell or progeny thereof.

In some embodiments, the method may include more than one differentiation step. Accoridngly, more than one differentiation medium may be employed. For example, a first differentiation medium may be used to initiate the differentiation of pluripotent stem cells into progenitor cells, followed by a step in which a second differentiation medium is used to expand and maintain the progenitor cells or to further differentiate the progenitor cells.

In some embodiments, the pluripotent stem cell comprises an embryonic stem cell or an embryo-derived cell. In some embodiments, the pluripotent stem cell comprises an induced pluripotent stem cell (iPSC). In some embodiments, the pluripotent stem cell is a human cell.

As used herein, the term “pluripotent stem cells” includes embryonic stem cells, embryo-derived stem cells, and induced pluripotent stem cells, regardless of the method by which the pluripotent stem cells are derived. Pluripotent stem cells are defined functionally as stem cells that are: (a) capable of inducing teratomas when transplanted in immunodeficient (SCID) mice; (b) capable of differentiating to cell types of all three germ layers (i.e., can differentiate to ectodermal, mesodermal, and endodermal cell types); and (c) express one or more markers of embryonic stem cells (e.g., express Oct 4, alkaline phosphatase, SSEA-3 surface antigen, SSEA-4 surface antigen, nanog, TRA-1-60, TRA-1-81, SOX2, REX1, etc.). Exemplary pluripotent stem cells can be generated using, for example, methods known in the art. Exemplary pluripotent stem cells include embryonic stem cells derived from the ICM of blastocyst stage embryos, as well as embryonic stem cells derived from one or more blastomeres of a cleavage stage or morula stage embryo (optionally without destroying the remainder of the embryo). Such embryonic stem cells can be generated from embryonic material produced by fertilization or by asexual means, including somatic cell nuclear transfer (SCNT), parthenogenesis, and androgenesis. Further exemplary pluripotent stem cells include induced pluripotent stem cells (iPS cells or iPSCs) generated by reprogramming a somatic cell by expressing a combination of factors (herein referred to as reprogramming factors). Induced pluripotent stem cells cells can be generated using fetal, postnatal, newborn, juvenile, or adult somatic cells. In some embodiments, factors that can be used to reprogram somatic cells to pluripotent stem cells include, for example, a combination of Oct4 (sometimes referred to as Oct 3/4), Sox2, c-Myc, and Klf4. In other embodiments, factors that can be used to reprogram somatic cells to pluripotent stem cells include, for example, a combination of Oct 4, Sox2, Nanog, and Lin28. In other embodiments, somatic cells are reprogrammed by expressing at least two reprogramming factors, at least three reprogramming factors, or at least four reprogramming factors. In other embodiments, additional reprogramming factors are identified and used alone or in combination with one or more known reprogramming factors to reprogram a somatic cell to a pluripotent stem cell. Induced pluripotent stem cells are defined functionally and include cells that are reprogrammed using any of a variety of methods (integrative vectors, non-integrative vectors, chemical means, etc.).

The pluripotent stem cells can be from any species. Embryonic stem cells have been successfully derived in, for example, mice, multiple species of non-human primates, and humans, and embryonic stem-like cells have been generated from numerous additional species. Thus, one of skill in the art can generate embryonic stem cells and embryo-derived stem cells from any species, including but not limited to, human, non-human primates, rodents (mice, rats), ungulates (cows, sheep, etc.), dogs (domestic and wild dogs), cats (domestic and wild cats such as lions, tigers, cheetahs), rabbits, hamsters, gerbils, squirrel, guinea pig, goats, elephants, panda (including giant panda), pigs, raccoon, horse, zebra, marine mammals (dolphin, whales, etc.) and the like. In some embodiments, the species is an endangered species. In some embodiments, the species is a currently extinct species.

Similarly, induced pluripotent stem cells can be from any species. Induced pluripotent stem cells have been successfully generated using mouse and human cells. Induced pluripotent stem cells have been successfully generated using embryonic, fetal, newborn, and adult tissue. Accordingly, one can readily generate induced pluripotent stem cells using a donor cell from any species. Thus, one can generate induced pluripotent stem cells from any species, including but not limited to, human, non-human primates, rodents (mice, rats), ungulates (cows, sheep, etc.), dogs (domestic and wild dogs), cats (domestic and wild cats such as lions, tigers, cheetahs), rabbits, hamsters, goats, elephants, panda (including giant panda), pigs, raccoon, horse, zebra, marine mammals (dolphin, whales, etc.) and the like. In some embodiments, the species is an endangered or currently extinct species.

Induced pluripotent stem cells can be generated using, as a starting point, virtually any somatic cell of any developmental stage. For example, the cell can be from an embryo, fetus, neonate, juvenile, or adult donor. Exemplary somatic cells that can be used include fibroblasts, such as dermal fibroblasts obtained by a skin sample or biopsy, synoviocytes from synovial tissue, foreskin cells, cheek cells, or lung fibroblasts. Although skin and cheek provide a readily available and easily attainable source of appropriate cells, virtually any cell can be used. In some embodiments, the somatic cell is not a fibroblast.

The pluripotent stem cells can be, for example, embryonic stem cells or induced pluripotent stem cells. Induced pluripotent stem cells can be produced by expressing a combination of reprogramming factors in a somatic cell. In some embodiments, at least two reprogramming factors are expressed in a somatic cell to successfully reprogram the somatic cell. In other embodiments, at least three reprogramming factors are expressed in a somatic cell to successfully reprogram the somatic cell. In other embodiments, at least four reprogramming factors are expressed in a somatic cell to successfully reprogram the somatic cell.

In some embodiments, the method may include steps of dispersing a pluripotent stem cell colony or clonal cell grouping to form dispersed essentially individual cells and seeding the dispersed cells into a culture that may contain a survival factor. For example, the cells may be seeded at a density of from about 10,000 stem cells per square centimeter of culturing surface to about 70,000 stem cells per square centimeter of culturing surface. In some embodiments, the cells may be seeded at a density of from about 10,000 stem cells per square centimeter of culturing surface to about 50,000 stem cells per square centimeter of culturing surface, or at a density of from about 20,000 stem cells per square centimeter of culturing surface to about 70,000 stem cells per square centimeter of culturing surface.

In some embodiments, the cells may be dispersed by mechanical or enzymatic means. For example, the cells may be dispersed by treatment with an effective amount of one or more enzymes, such as trypsin or trypLE, or a mixture of enzymes such as Accutase®.

In some embodiments, the method may include steps of seeding the pluripotent stem cells in a culturing medium, which may contain a matrix component and/or a survival factor, to form a culture; introducing a differentiation medium into the culture, wherein the differentiation medium is free or essentially free of feeder cells and includes at least one recombinant growth factor selected from the group consisting of BMP-4, VEGF, and bFGF; and differentiating the cells under a hypoxic atmosphere having less than about 5.5% oxygen for a period of time sufficient to generate progenitor cells. In some embodiments, one or more of these steps may be employed to produce CD34+ progenitor cells, CD31+ progenitor cells, CD43+ progenitor cells, or CD34+CD43+ progenitor cells. The progenitor cells may then be harvested, and they may further be sorted. At this point, the progenitor cells may be maintained, expanded, or further differentiated.

In some embodiments, the one or more culture media may include a growth factor. Examples of growth factors may include, but are not limited to, BMP-4, VEGF, bFGF, stem cell factor (SCF), Flt-3 ligand, interleukin 3 (IL-3), interleukin 6 (IL-6), interleukin 9 (IL-9), interleukin 11 (IL-11), insulin-related growth factor 1 (IFG1), insulin-related growth factor 2 (IGFII), thrombopoietin (TPO), granulocyte-macrophage-colony-stimulating factor (GM-CSF), and granulocyte colony-stimulating factor (G-CSF). The one or more culture media may include one, two, three, or more of these growth factors; for example, other growth factors may be included in a defined medium in order to increase proliferation or modulate the differentiation state of the cells. Various amounts of these factors may be used to stimulate cellular responses (e.g., in the amounts described in Yamamura et al., 2007; Fadilah et al., 2007; Bashey et al., 2007).

In some embodiments, the one or more culture media may include a supplement selected from inositol, folic acid, monothioglycerol, transferrin, insulin, ferrous nitrate, ferrous sulfate, BSA, L-glutamine, penicillin-streptomycin, and combinations thereof.

In some embodiments, the one or more culture media comprise a serum-free culture medium or a culture medium substantially free of serum. In some embodiments, the one or more culture media comprise a defined differentiation medium.

As used herein, the term “serum-free culture medium” refers to a culture medium that has not been supplemented with animal serum. The serum-free medium is a known composition medium, but the serum-free medium can be supplemented with individual animal or plant proteins, or protein fractions. The term “serum,” as used herein, refers to a non-human animal product that may be added to a culture to provide nutrients to growing cells.

As used herein, the terms “defined conditions,” “defined medium,” and “defined differentiation” refer to culture conditions, wherein the culture has known quantities of all ingredients and does not utilize undefined ingredients, serum, or feeder cells (e.g., mouse embryonic fibroblasts). An “undefined ingredient” is an ingredient that contains unknown components, or contains known components in unknown amounts. Defined conditions may be particularly useful, e.g., in applications where differentiated cells may be therapeutically administered to a subject, such as a human patient.

In some embodiments, the differentiation medium may include a survival factor. The survival factor may be, for example, an inhibitor of a Rho-associated kinase (ROCK), such as HA100 or H1152, or an inhibitor of myosin II, such as blebbistatin.

In some embodiments, the method comprises supplementing the one or more culture media with a cytokine only at the early stage of culturing, e.g., from day 0 to day 17 (e.g., from day 0 to day 1, from day 0 to day 2, from day 0 to day 3, from day 0 to day 4, from day 0 to day 5, from day 0 to day 6, from day 0 to day 7, from day 0 to day 8, from day 0 to day 9, from day 0 to day 10, from day 0 to day 11, from day 0 to day 12, from day 0 to day 13, from day 0 to day 14, from day 0 to day 15, from day 0 to day 16, from day 0 to day 18). The advantages of only supplementing the one or more culture media with a cytokine at the early stage of culturing include significantly reduced overall costs to produce red blood cells, thus enabling large-scale production of red blood cells for therapeutic use.

In some embodiments, the method further comprises expanding the pluripotent stem cell prior to being differentiated into the red blood cell.

The term “culturing” or “expanding” refers to maintaining or cultivating cells under conditions in which they can proliferate and avoid senescence. For example, cells may be cultured in media optionally containing one or more growth factors, i.e., a growth factor cocktail. In some embodiments, the cell culture medium is a defined cell culture medium. Stable cell lines may be established to allow for the continued propagation of cells.

In some embodiments, a robot may be employed to automate at least a portion of the disclosed method. For example, a plurality of the human embryonic stem cells may be cultured using a bioreactor (e.g., a hollow fiber bioreactor). For example, one or more steps for the culture of stem cells and/or differentiation of progenitor cells from pluripotent stem cells may be automated. Automating a process using robotic or other automation can allow for more efficient and economical methods for the production, culture, and differentiation of cells. For example, robotic automation may be utilized as described in U.S. patent application No. 20090029462, incorporated herein by reference in its entirety.

A bioreactor may also be used to culture, maintain, and/or differentiate cells (e.g., human embryonic stem cells, CD34+ cells, CD31+ cells, hematopoietic cells, etc.) according to the present disclosure. Bioreactors provide the advantage of allowing for the “scaling up” of a process in order to produce an increased amount of cells. Various bioreactors may be used with this disclosure, including batch bioreactors, fed batch bioreactors, continuous bioreactors (e.g., a continuous stirred-tank reactor model), and/or a chemostat. Pluripotent stem cells may be cultured on the robot, using flat plates in order to induce differentiation into CD34/43+ cells. Once separation of the cells has occurred, spinner flasks or a bioreactor may be used to generate large numbers of cells. Robotics may include liquid handling tools such as cap-piercing probes and disposable tips to minimize carry-over between samples. In some embodiments, robotics may be utilized in conjunction with one or more bioreactors for culturing cells (e.g., during the maintenance or growth of pluripotent stem cells, the differentiation of pluripotent stem cells into red blood cells, etc.).

In some embodiments, the method further comprises differentiating the pluripotent stem cell into a progenitor cell and sorting a population of progenitor cells differentiated from the pluripotent stem cell using magnetic-activated cell sorting (MACS), flow cytometry, or fluorescence-activated cell sorting (FACS). In some embodiments, the method further comprises sorting the population of progenitor cells based on the expression of one or more of CD31, CD34, CD43, and CD45.

A “progenitor cell,” as used herein, refers to a lineage-committed cell derived from a pluripotent stem cell. Thus, progenitor cells are more differentiated than pluripotent stem cells, but still have the capacity to differentiate into more than one type of cell. For example, a hematopoietic progenitor cell is more differentiated than a pluripotent stem cell, but the hematopoietic progenitor cell still has the capacity to differentiate into, for example, an erythrocyte, a macrophage, a granulocyte, a megakaryocyte, a dendritic cell, or a mast cell. In some embodiments, the progenitor cell is a hematopoietic progenitor cell. In yet other embodiments, the progenitor cell is a hematoendothelial (or hemangioblast) progenitor cell, which is capable of differentiating into hematopoietic cells or endothelial cells.

### Red Blood Cells (RBCs)

In another aspect, this disclosure also provides blood, cellular and acellular blood components, or blood products obtained from the red blood cell produced by the method disclosed herein.

In some embodiments, the red blood cell expresses ADAMTS13, asparaginase, Factor VIII, Factor IX, or phenylalanine hydroxylase. For example, the pluripotent stem cell may be further genetically modified such that the the red blood cell expresses ADAMTS13, asparaginase, Factor VIII, Factor IX, or phenylalanine hydroxylase.

An “ADAMTS13,” as used herein, refers to any protein or polypeptide with ADAMTS13 activity, particularly the ability to cleave the peptide bond between residues Tyr-842 and Met-843 of a von Willebrand factor (VWF). For example, an ADAMTS13 protein may be a polypeptide comprising an amino acid sequence having significant identity to that of NP_620594 (ADAMTS13 isoform 1, preproprotein) or amino acids 75 to 1427 of NP_620594 (ADAMTS13 isoform 1, mature polypeptide).

An “asparaginase,” as used herein, refers to an enzyme that has asparaginase activity, that is, an enzyme that catalyzes the hydrolysis of asparagine to obtain aspartic acid. As a medication, L-asparaginase is used to treat acute lymphoblastic leukemia.

A “phenylalanine hydroxylase (PAH),” as used herein, refers to an enzyme that catalyzes the hydroxylation of the aromatic side-chain of phenylalanine to generate tyrosine. PAH is one of three members of the biopterin-dependent aromatic amino acid hydroxylases, a class of monooxygenase that uses tetrahydrobiopterin and a non-heme iron for catalysis. Deficiency in PAH activity due to mutations in PAH causes hyperphenylalaninemia (HPA), and when blood phenylalanine levels increase above 20 times the normal concentration, the metabolic disease phenylketonuria (PKU) results.

A “factor VIII,” as used herein, refers to an essential blood-clotting protein, also known as an anti-hemophilic factor. In humans, factor VIII is encoded by the F8 gene. Defects in this gene result in hemophilia A, a recessive X-linked coagulation disorder.

A “Factor IX,” as used herein, refers to a serine protease of the coagulation system that helps the blood form clots to stop bleeding. It belongs to the peptidase family S1. Deficiency of this protein causes hemophilia B. Injections of Factor IX can be used to treat hemophilia B, which is also called Christmas disease, a condition in which the body does not make enough factor IX.

In some embodiments, the red blood cell may be irradiated to kill any live cells that might be present in the preparation. In some embodiments, red blood cells have been irradiated with at least 2500 cGy of radiation.

In some embodiments, red blood cells may be modified by loading with (or conjugation with) one or more molecular agents, such as a therapeutic agent. Such molecular agents can be internalized within the red blood cell and may include, but are not limited to, a compound that is configured to provide an activity to the subject and/or to the red blood cell following administration. In some embodiments, such molecular agents may include, but are not limited to, one or more therapeutic agents or imaging agents.

In some embodiments, the molecular agent can be a therapeutic agent, such as a small molecule drug or biological effector molecule. Therapeutic agents of interest include, without limitation, pharmacologically active drugs, genetically active molecules, etc. Therapeutic agents of interest include antineoplastic agents, anti-inflammatory agents, hormones or hormone antagonists, ion channel modifiers, and neuroactive agents.

Small molecules, including inorganic and organic chemicals, may also be used. In an embodiment, the small molecule is a pharmaceutically active agent. Useful classes of pharmaceutically active agents include, but are not limited to, antibiotics, anti-inflammatory drugs, angiogenic or vasoactive agents, growth factors, and chemotherapeutic (antineoplastic) agents (e.g., tumor suppressors).

If a prodrug is loaded in an inactive form, a second effector molecule may be loaded into a modified red blood cell or a red blood cell that is to be modified according to the disclosure herein. Such a second effector molecule is usefully an activating polypeptide that converts the inactive prodrug to active drug form. In an embodiment, activating polypeptides include, but are not limited to, viral thymidine kinase, carboxypeptidase A, α-galactosidase, β-glucuronidase, alkaline phosphatase, or cytochrome P-450, plasmin, carboxypeptidase G2, cytosine deaminase, glucose oxidase, xanthine oxidase, β-glucosidase, azoreductase, t-glutamyl transferase, β-lactamase, or penicillin amidase. Either the polypeptide or the gene encoding it may be loaded into the modified, or to-be-modified, red blood cells; if the latter, both the prodrug and the activating polypeptide may be encoded by genes on the same recombinant nucleic acid construct. Furthermore, either the prodrug or the activator of the prodrug may be already loaded into the red blood cell. The relevant activator or prodrug (as the case may be) is then loaded as a second agent according to the methods described herein.

The therapeutic agent may also be a biological effector molecule that has activity in a biological system. Biological effector molecules, include but are not limited to, a protein, polypeptide, or peptide, including, but not limited to, a structural protein, an enzyme, a cytokine (such as an interferon and/or an interleukin), a polyclonal or monoclonal antibody, or an effective part thereof, such as an Fv fragment, which antibody or part thereof, may be natural, synthetic or humanized, a peptide hormone, a receptor, or a signaling molecule. Included within the term “immunoglobulin” are intact immunoglobulins as well as antibody fragments such as Fv, a single chain Fv (scFv), a Fab or a F (ab′)₂.

The biological effector molecules can be immunoglobulins, antibodies, Fv fragments, etc., that are capable of binding to antigens in an intracellular environment. These types of molecules are known as “intrabodies” or “intracellular antibodies.” An “intracellular antibody” or an “intrabody” includes an antibody that is capable of binding to its target or cognate antigen within the environment of a cell, or in an environment that mimics an environment within the cell. Selection methods for directly identifying such “intrabodies” include the use of an in vivo two-hybrid system for selecting antibodies with the ability to bind to antigens inside mammalian cells. Such methods are described in PCT/GB00/00876, incorporated herein by reference.

The biological effector molecule includes, but is not limited to, at least one of a protein, a polypeptide, a peptide, a nucleic acid, a virus, a virus-like an amino acid, an amino acid analog, a modified amino acid, a modified amino acid analog, a steroid, a proteoglycan, a lipid, and a carbohydrate or a combination thereof (e.g., chromosomal material comprising both protein and DNA components or a pair or set of effectors, wherein one or more convert another to an active form, for example catalytically). A biological effector molecule may include a nucleic acid, including, but not limited to, an oligonucleotide or modified oligonucleotide, an antisense oligonucleotide or modified antisense oligonucleotide, an aptamer, a cDNA, genomic DNA, an artificial or natural chromosome (e.g., a yeast artificial chromosome) or a part thereof, RNA, including a siRNA, a shRNA, mRNA, tRNA, rRNA or a ribozyme, or a peptide nucleic acid (PNA); a virus or virus-like particles; a nucleotide or ribonucleotide or synthetic analog thereof, which may be modified or unmodified.

The biological effector molecule can also be an amino acid or analog thereof, which may be modified or unmodified or a non-peptide (e.g., steroid) hormone; a proteoglycan; a lipid; or a carbohydrate. If the biological effector molecule is a polypeptide, it can be loaded directly into a modified red blood cell, according to the methods described herein. Alternatively, a nucleic acid molecule bearing a sequence encoding a polypeptide, which sequence is operatively linked to transcriptional and translational regulatory elements active in a cell at a target site, may be loaded.

The molecular agent may be an imaging agent, which may be detected, whether in vitro or in vivo in the context of a tissue, organ or organism. Examples of agents include those useful for imaging of tissues in vivo or ex vivo. For example, imaging agents, such as labeled antibodies which are specific for defined molecules, tissues or cells in an organism, may be used to image specific parts of the body by releasing from the loaded red blood cells at a desired location using electromagnetic radiation. In some embodiments, the imaging agent emits a detectable signal, such as visible light or other electromagnetic radiation. The imaging agent can be a radioisotope, e.g., ³²P or ³⁵S or ⁹⁹Tc, or a quantum dot, or a molecule such as a nucleic acid, polypeptide, or other molecules, conjugated with such a radioisotope. The imaging agent can be opaque to radiation, such as X-ray radiation. In another embodiment, the imaging agent comprises a targeting functionality by which it is directed to a particular cell, tissue, organ or other compartments within the body of an animal. For example, the agent may comprise a radiolabelled antibody that specifically binds to defined molecule(s), tissue(s) or cell(s) in an organism.

The modified red blood cells may also be labeled with one or more positive markers that can be used to monitor over time the number or concentration of modified red blood cells in the blood circulation of an individual. It is anticipated that the overall number of modified red blood cells will decay over time following initial transfusion. As such, it may be appropriate to correlate the signal from one or more positive markers with that of the activated molecular marker, generating a proportionality of signal that will be independent of the number of modified red blood cells remaining in the circulation. There are presently several fluorescent compounds, for example, that are approved by the Food & Drug Administration for human use, including but not limited to fluorescein, indocyanine green, and rhodamine B. For example, red blood cells may be non-specifically labeled with fluorescein isothiocyanate.

### Compositions

Also within the scope of this disclosure is a composition comprising the red blood cell produced by the method described herein, or the blood, cellular and acellular blood components, or blood products described herein.

In some embodiments, the modified red blood cells as described above can also be incorporated into pharmaceutical compositions suitable for administration.

The pharmaceutical compositions generally comprise substantially purified modified red blood cells and a pharmaceutically acceptable carrier in a form suitable for administration to a subject. Pharmaceutically-acceptable carriers are determined in part by the particular composition being administered, as well as by the particular method used to administer the composition. The pharmaceutical compositions are generally formulated as sterile, substantially isotonic and in full compliance with all Good Manufacturing Practice (GMP) regulations of the U.S. Food and Drug Administration.

The terms “pharmaceutically acceptable,” “physiologically tolerable,” as referred to compositions, carriers, diluents, and reagents, are used interchangeably and include materials are capable of administration to or upon a subject without the production of undesirable physiological effects to the degree that would prohibit administration of the composition. For example, “pharmaceutically-acceptable excipient” includes an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

Examples of such carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the modified red blood cells, use thereof in the compositions is contemplated. Supplementary active compounds can also be incorporated into the compositions.

A pharmaceutical composition is formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate-buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. In many cases, it will be preferable to include isotonic compounds, e.g., sugars, polyalcohols such as mannitol, sorbitol, sodium chloride in the composition. Prolonged absorption of the injectable compositions can be brought about by including in the composition a compound that delays absorption, e.g., aluminum monostearate and gelatin.

Sterile injectable solutions can be prepared by incorporating the modified red blood cells in the required amount in an appropriate solvent with one or a combination of ingredients enumerated above, as required. Generally, dispersions are prepared by incorporating the modified red blood cells into a sterile vehicle that contains a basic dispersion medium and the required other ingredients from those enumerated above. In the case of sterile powders for the preparation of sterile injectable solutions, methods of preparation are vacuum drying and freeze-drying which yields a powder of the active ingredient plus any additional desired ingredient from a previously sterile-filtered solution thereof. The modified red blood cells can be administered in the form of a depot injection or implant preparation which can be formulated in such a manner as to permit a sustained or pulsatile release of the active ingredient.

Systemic administration can also be by transmucosal or transdermal means. For transmucosal or transdermal administration, penetrants appropriate to the barrier to be permeated are used in the formulation. Such penetrants are generally known in the art, and include, e.g., for transmucosal administration, detergents, bile salts, and fusidic acid derivatives. For transdermal administration, the modified red blood cells are formulated into ointments, salves, gels, or creams, as generally known in the art.

In some embodiments, the modified red blood cells are prepared with carriers that will protect the modified red blood cells against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically-acceptable carriers.

In some embodiments, the composition includes the red blood cells as described above and optionally a cryo-protectant (e.g., glycerol, DMSO, PEG).

### Kits

In yet another aspect, this disclosure provides a kit comprising the red blood cell produced by the method described herein, the blood, cellular and acellular blood components, or blood products, or the composition, as described herein. In some embodiments, the kit comprises the modified red blood cells or the composition described above.

Also within the scope of this disclosure is a kit for producing red blood cells from an induced pluripotent stem cell. In some embodiments, the kit may include a genetically modified pluripotent stem cell comprising a constitutively active SCF receptor. In some embodiments, the kit may further include a medium free of SCF and/or erythropoietin (Epo) for culturing the pluripotent stem cell and differentiating the pluripotent stem cell into a red blood cell.

For example, a kit may comprise a differentiation medium described herein in one or more sealed vials. The kit may include a cell, such as a pluripotent stem cell, progenitor cell, or hematopoietic progenitor cell. Suitable kits include various reagents for use in suitable containers and packaging materials, including tubes, vials, and shrink-wrapped and blow-molded packages.

In some embodiments, the kit may further include materials including, but not limited to, one or more of the following: a matrix component, fibronectin, collagen, an RGD peptide, BIT 9500, BMP4, VEGF, bFGF, L-glutamine, non-essential amino acids, monothioglycerol, penicillin, streptomycin, an inhibitor of a Rho-associated kinase (ROCK), an inhibitor of myosin II, amino acids, TeSR medium, TeSR2 medium, mTeSR medium, enzymes, trypsin, trypLE, antibiotics, vitamins, salts, minerals, or lipids.

The kit may also include instructions for producing progenitor cells (e.g., hematopoietic progenitor cells) and red blood cells. The kit may further include instructions for administrating the modified red blood cells or the composition and optionally an adjuvant.

### Methods of Use

This disclosure further provides methods of treating a disease, disorder, or injury by administering to a subject a pharmaceutically effective amount of red blood cells obtained by the methods disclosed herein. Administration of these compositions will be via any common route so long as the target tissue is available via that route. This includes administration by systemic or parenteral methods, including intravenous injection.

Diseases or disorders that may be treated by methods disclosed here include, but are not limited to, a vascular disease or disorder, an immunological disease or disorder, a neuronal disease or disorder, a blood disease or disorder, or an injury. For example, hematopoietic progenitor cells may be differentiated into red blood cells to be used in blood transfusions. One important application is production of red blood cells for transfusion purpose, particularly of cells carrying a rare blood group that are difficult to procure (for instance, to treat allo-immunized sickle cell patients). Similarly, the cells can be used to generate reagent red blood cells that are used by blood banks to cross match the patient and the cells to be transfused and to help identified antibodies against some blood groups that are present in some patients (so-called allo-immunized patients).

The red blood cells can be administered by infusion. In some embodiments, the method may include producing the red blood cells in vitro by the disclosed methods before administrating them to the subject. In some embodiments, the red blood cells can be produced in a bioreactor, e.g., a hollow fiber culturing system. The red blood cells can be administered to individuals through infusion or injection (for example, intravenous) or other methods known in the art. Administration may be once every two weeks, once a week, or more often, but the frequency may be decreased during a maintenance phase of the disease or disorder.

The red blood cells may be administered in a pharmaceutical formulation as described above. The dose of the red blood cells for an optimal therapeutic benefit can be determined clinically. A certain length of time is allowed to pass for the circulating or locally delivered modified red blood cells. The waiting period will be determined clinically and may vary depending on the composition of the composition. For example, the dose and the administration frequency will depend on the clinical signs, which confirm maintenance of the remission phase, with the reduction or absence of at least one or more preferably more than one clinical signs of the acute phase known to the person skilled in the art. More generally, dose and frequency will depend in part on the recession of pathological signs and clinical and subclinical symptoms of a disease condition or disorder contemplated for treatment with the above-described composition. Dosages and administration regimens can be adjusted depending on the age, sex, physical condition of administered, as well as the benefit of the treatment and side effects in the patient or mammalian subject to be treated and the judgment of the physician, as is appreciated by those skilled in the art. In all of the above-described methods, the cells can be administered to a subject at 1×10⁴ to 1×10¹⁰/time.

### Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, by disulfide bond formation, glycosylation, lipidation, acetylation, phosphorylation, or any other manipulation, such as conjugation with a labeling component. As used herein, the term “amino” acid” includes natural and/or unnatural or synthetic amino acids, including glycine and both the D or L optical isomers, and amino acid analogs and peptidomimetics.

The term “amino acid sequence” refers to an amino acid sequence of a protein molecule, “amino acid sequence” and like terms, such as “polypeptide” or “protein,” are not meant to limit the amino acid sequence to the complete, native amino acid sequence associated with the recited protein molecule. Furthermore, an “amino acid sequence” can be deduced from the nucleic acid sequence encoding the protein.

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene products.” If the polynucleotide is derived from genomic DNA, expression may include splicing of the mRNA in a eukaryotic cell.

As used herein, the term “subject” refers to a vertebrate, and in some exemplary aspects, a mammal. Such mammals include, but are not limited to, mammals of the order Rodentia, such as mice and rats, and mammals of the order Lagomorpha, such as rabbits, mammals from the order Carnivora, including Felines (cats) and canines (dogs), mammals from the order Artiodactyla, including bovines (cows) and swines (pigs) or of the order Perissodactyla, including Equines (horses), mammals from the order Primates, Ceboids, or Simoids (monkeys) and of the order Anthropoids (humans and apes). In exemplary aspects, the mammal is a mouse. In more exemplary aspects, the mammal is a human.

The term “disease” as used herein is intended to be generally synonymous and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, “treatment,” “treating,” “palliating,” and “ameliorating” are used interchangeably. These terms refer to an approach for obtaining beneficial or desired results, including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

As used herein, the term “administering” refers to the delivery of cells by any route, including intravenous administration.

As used herein, the term “effective amount” or “therapeutically effective amount” refers to an amount which results in measurable amelioration of at least one symptom or parameter of a specific disorder. A therapeutically effective amount of the above-described cells can be determined by methods known in the art. An effective amount for treating a disorder can be determined by empirical methods known to those of ordinary skill in the art. The exact amount to be administered to a patient will vary depending on the state and severity of the disorder and the physical condition of the patient. A measurable amelioration of any symptom or parameter can be determined by a person skilled in the art or reported by the patient to the physician. It will be understood that any clinically or statistically significant attenuation or amelioration of any symptom or parameter of the above-described disorders is within the scope of this disclosure. Clinically significant attenuation or amelioration means perceptible to the patient and/or to the physician.

Doses are often expressed in relation to bodyweight. Thus, a dose which is expressed as [g, mg, or other unit]/kg (or g, mg etc.) usually refers to [g, mg, or other unit] “per kg (or g, mg etc.) bodyweight”, even if the term “bodyweight” is not explicitly mentioned.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

The terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

“Combination” therapy, as used herein, unless otherwise clear from the context, is meant to encompass administration of two or more therapeutic agents in a coordinated fashion and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on administration of another therapeutic agent. For example, one therapeutic agent may be administered only after a different therapeutic agent has been administered and allowed to act for a prescribed period of time. See, e.g., Kohrt et al. (2011) Blood 117:2423.

As used herein, the term “pharmaceutical composition” refers to a mixture of at least one compound useful within the invention with other chemical components, such as carriers, stabilizers, diluents, dispersing agents, suspending agents, thickening agents, and/or excipients. The pharmaceutical composition facilitates administration of the compound to an organism.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

The term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of this disclosure within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition that is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

It is to be understood that wherever values and ranges are provided herein, all values and ranges encompassed by these values and ranges, are meant to be encompassed within the scope of this disclosure. Moreover, all values that fall within these ranges, as well as the upper or lower limits of a range of values, are also contemplated by the present application.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. When used in this document, the term “exemplary” is intended to mean “by way of example” and is not intended to indicate that a particular exemplary item is preferred or required.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of this disclosure. Nothing herein is to be construed as an admission that this disclosure is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1

This example describes the materials and methods used in the following examples.

Induced-pluripotent stem cells (iPSC): iPSC were reprogrammed from peripheral blood mononuclear cells using the Sendai virus approach (CytoTune-iPS 2.0 Sendai Reprogramming Kit—Thermo Fisher Scientific) according to the manufacturer's instruction.

Pluripotent stem cell culture: human pluripotent stem cells (hPSC) were maintained undifferentiated in an E8 medium (Chen G, et al. Nat Methods. 2011 May; 8 (5): 424-9. PMID: 21478862) on Vitronectin (Life Technologies) and passaged using EDTA every 3-4 days depending on their confluence stage.

Pluripotent stem cell transfection: hPSC were treated with ROCK inhibitor (10 μM) at least 3 hours prior to detachment with Accutase. Single cell suspension was achieved mechanically by pipetting up and down. Aliquots of 25.10+ cells were resuspended in electroporation buffer (PBS with MgCl2 11.6 mM) and mixed with 4 μg of sgRNA, 4 μg of donor DNA, and 1 μg of cas9mRNA. The mix was then electroporated with a NEPA21 electroporator (Nepagene) with two 10 ms poring pulses of 100V 50 ms apart and five 50 ms transfer pulses of 40V 50 ms apart. After electroporation, the cells were plated on vitronectin in E8 containing ROCK inhibitor (10 μM) for 24 hours before reverting to E8 without ROCK inhibitor.

Differentiation media: IMIT (Olivier et al.), an IMDM-based media that contains insulin (10 μg/ml), transferrin (200 μg/ml), lipids (1/200), ethanolamine (1 mM), Trolox (a water-soluble form of vitamin E, 50 μM), and methyl-β-cyclodextrin (0.1 mg/ml), but no albumin

R6 (Olivier E N, Exp Hematol. 2019 July; 75:31-52.e15), a RPMI-based media that contains L-ascorbic acid (220 μM), Insulin (10 μg/ml), Recombinant Transferrin (20 μg/ml), Lipids (1/200), and FeIII-EDTA (4 μM).

Differentiation of iPSC into Erythroid Cells: Albumin-Free Protocol:

On day-1: Three-day-old hPSC colonies were dissociated with 5 mM EDTA in PBS for 6 minutes. The EDTA was then removed and replaced with 5 mL of E8 medium, and the well was thoroughly flushed with a 5 mL serological pipet. Small clumps were generated to produce small colonies of about 50 cells on day 0. The cells were then plated at 100-200×10³/well in 2 mL/well of E8 medium on vitronectin in tissue culture treated six-well plates (Falcon), which are used throughout the protocol. After plating, the cells were allowed to attach overnight.

### Short PSC-RED Protocol

On day 0: Differentiation was induced by replacing the E8 medium with IMIT medium, containing supplement 1 (Bone Morphogenic Protein 4 (BMP4) (10 ng/ml), Vascular Endothelium Growth Factor 165 (VEGF) (10 ng/mL), basic Fibroblast Growth Factor (bFGF) (10 ng/ml), Wnt3A (5 ng/mL), Wnt5A (5 ng/ml), Activin A (5 ng/ml) and Inhibitor VIII (2 μM))

Before inducing the differentiation, the culture was inspected to ascertain that the colonies contained about 50 cells. One well of the culture was sacrificed for cell counting in order to calculate the yield of cells at the end of the experiments.

On day 2, 6× concentrated supplement 2 in IMIT was added to each well to bring the final concentration of fresh cytokines to 20 ng/ml of BMP-4, 30 ng/mL of VEGF, 5 ng/ml of Wnt3A, 5 ng/ml of Wnt5A, 5 ng/ml of Activin A, 2 μM of Inhibitor VIII, 10 ng/ml of bFGF, 20 ng/mL of Stem Cell Factor (SCF) and 0.4 ng/ml of α-Estradiol.

On day 3, the cells were dissociated with TrypleSelect 1× for 5-10 minutes at 37 C. After the addition of 10 mL of PBS, cells were centrifuged for 3 minutes at 250 g, the supernatant was discarded and the cells re-suspended in fresh IMIT medium containing supplement 3 (BMP4 (20 ng/ml), VEGF (30 ng/ml), bFGF (20 ng/mL), SCF (30 ng/mL), Insulin-like Growth Factor 2 (IGF2) (10 ng/mL), Thrombopoietin (TPO) (10 ng/mL), SB431542 (3 μM), Heparin (5 μg/mL), Iso Butyl Methyl Xanthine (IBMX) (50 μM) and α-Estradiol (0.4 ng/mL) and plated at 1.10⁵ cells/mL of a tissue culture treated six wells plate (3 mL per well).

On day 6, the cells were centrifuged for 3 minutes at 350 g and re-suspended at 5.10⁵/mL in fresh IMIT medium containing supplement 3 without SB431542 but with 30 nM of UM171.

Between day 6 and day 10, the cell concentration was maintained below 1.510⁶ cells/mL by dilution to 0.5.10⁶/mL as needed in the same medium and supplement, and a fresh dose of 6× concentrated supplement was added at day 8 to fully renew the cytokines and small molecules.

On day 10, the cells were centrifuged for 3 minutes at 250 g, plated at 0.66·10⁵ cells/mL in IMIT containing the SED supplement (SCF 100 ng/mL, Erythropoietin 4 U/mL, IBMX 50 μM, and Dexamethasone 1 μM). From day 10 to day 17 the cell concentration was maintained below 1.5·10⁶/mL by dilution in IMIT plus the SED supplement, and 6× concentrated SED supplement in IMIT was added every 2 days to fully renew the cytokines and small molecules.

On day 17, the cells were centrifuged for 3 minutes at 250 g and plated at a density of about 2×10⁵/mL of IMIT containing the SER supplement (SCF (50 ng/ml), Erythropoietin (EPO) (4 U/mL), and RU486 (1 μM)). From day 17 to 24, the cell concentration was maintained below 1.5·10⁶/mL by dilution in IMIT plus SER supplement, and 6× concentrated SER supplement in IMIT was added every 2 days to fully renew the cytokines and small molecules.

On day 24, the cells were centrifuged for 3 minutes at 250 g and plated at 2×10⁵/mL in R5 medium with the SER2 supplement (SCF (10 ng/mL), Erythropoietin (EPO) (4 U/mL), and RU486 (1 μM)). From day 24 to 31, the cell concentration was maintained below 1.5·10⁶/mL by dilution in R % plus the SER 2 supplement, and 6× concentrated SER2 supplement in R5 was added every 2 days to fully renew the cytokines and small molecules.

On day 31, the cells were centrifuged for 3 minutes at 250 g and maintained in R5 medium alone for up to 8 days.

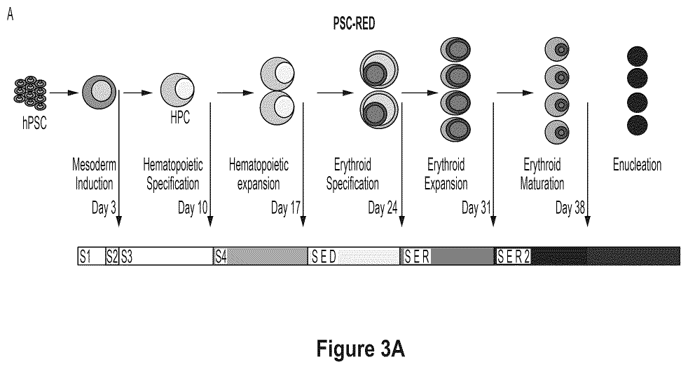

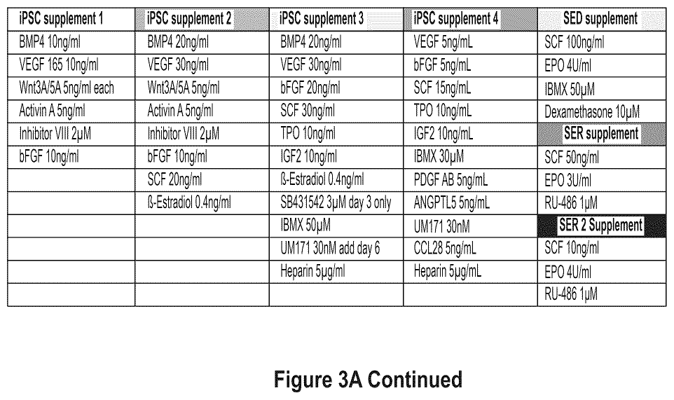

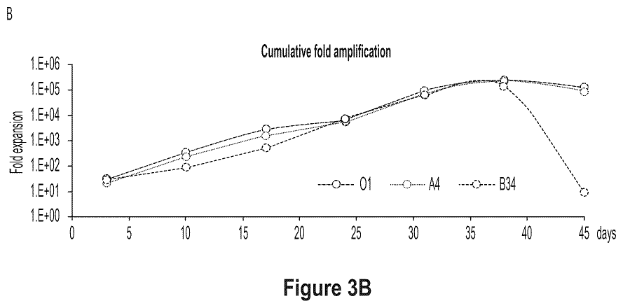

TABLE 1
Components of supplements as used in the protocol (also see FIG. 3A)

| iPSC | iPSC | iPSC | iPSC | SED |
|---|---|---|---|---|
| supplement 1 | supplement 2 | supplement 3 | supplement 4 | supplement |
| BMP4 10 ng/ml | BMP4 20 ng/ml | BMP4 20 ng/ml | VEGF 5 ng/mL | SCF 100 ng/ml |
| VEGF 165 | VEGF 30 ng/ml | VEGF 30 ng/ml | bFGF 5 ng/mL | EPO 4 U/ml |
| 10 ng/ml | | | | |
| Wnt3A/5A | Wnt3A/5A | bFGF 20 ng/ml | SCF 15 ng/mL | IBMX 50 mM |
| 5 ng/ml each | 5 ng/ml | | | |
| Activin A 5 ng/ml | Activin A | SCF 30 ng/ml | TPO 10 ng/mL | Dexamethasone |
| | 5 ng/ml | | | 10 mM |
| Inhibitor VIII | Inhibitor VIII | TPO 10 ng/ml | IGF2 10 ng/mL | SER supplement |
| 2 μM | 2 μM | | | |
| bFGF 10 ng/ml | bFGF 10 ng/ml | IGF2 10 ng/ml | IBMX 30 μM | SCF 50 ng/ml |
| | SCF 20 ng/ml | b-Estradiol | PDGF AB | EPO 3 U/ml |
| | | 0.4 ng/ml | 5 ng/mL | |
| | b-Estradiol | SB431542 3 μM | ANGPTL5 | RU-486 1 μM |
| | 0.4 ng/ml | day 3 only | 5 ng/mL | |
| | | IBMX 50 μM | UM171 30 nM | SER 2 |
| | | | | Supplement |
| | | UM171 30 nM add | CCL28 5 ng/mL | SCF 10 ng/ml |
| | | day 6 | | |
| | | Heparin 5 μg/ml | Heparin | EPO 4 U/ml |
| | | | 5 μg/mL | |
| | | | | RU-486 1 μM |

### Long Differentiation Protocol

This long protocol is identical to the short protocol but an additional HSC maintenance and amplification step is added after day 10. This step consist on centrifuging the cells at 250 g for three minutes and replating the day 10 cells in IMIT at 2.10⁵/mL in the presence of supplement 4 (bFGF (5 ng/ml), SCF (15 ng/ml), VEGF (5 ng/mL), TPO (10 ng/ml), IGF2 (10 ng/ml), Platelet Derived Growth Factor (PDGF) (5 ng/mL), Angiopoietin-like 5 (Angpt15) (5 ng/ml), CCL28 (5 ng/mL), IBMX 30 M, Heparin (5 μg/mL) and UM171 (30 nM) for one or two weeks. As mentioned above, the concentration of cells was kept below 1.5·10⁶ cells/mL at all times, and cytokines were refreshed every two days by adding 6× concentrated supplement. Cells, kept for two weeks under these conditions, were centrifuged and replated on fresh plates after 7 days to eliminate any attached cells.

After this additional step, the differentiation resumed according to the short protocol day 10. A one-time addition of Granulocyte-Macrophage Colony Stimulating Factor (GMCSF) (20 ng/mL) and Granulocyte Stimulating Factor (GCSF) (20 ng/mL) was, however, necessary to induce maximal proliferation of the stem and progenitor cells in the SED supplements.

### Analysis and Characterization

Cell enumeration: Cells were counted with a Luna-FL dual channel Automated Cell Counter (Logos) using acridine orange to visualize the live cells and propidium iodide to exclude the dead cells

Flow cytometry: iPSC undergoing differentiation were evaluated by FACS using antibodies against CD31, CD34, CD36, CD41a, CD43, CD45, CD71, and CD235a, also known as glycophorin A (BD Biosciences and eBioscience). MNC undergoing differentiation were evaluated by FACS using CD36, CD71, and CD235a antibodies.

Enucleation: The enucleation rate was measured using the DRAQ5 DNA nuclear stain (ThermoFisher) after exclusion of dead cells with Propidium Iodide. The cells were analyzed with an Aurora flow cytometer (Cytek) flow cytometer, and the flow cytometry data were analyzed with the Flowjo software.

Giemsa staining: Erythroid differentiation and enucleation were also assessed microscopically by Rapid Romanovsky staining of cytospin preparations. Cell sizes were estimated on a Nikon TE-2000S microscope using software provided by the manufacturer.

RBC filtration: To eliminate the nuclei at the end of the experiments, RBC were filtered using PAL Acrodisc 25 mm WBC filters as recommended by the manufacturer. Filtered cRBC were stored for up to one month with little signs of hemolysis in Alsever's solution (Sigma).

HPLC analysis: Cells were washed twice with PBS, and lysed in water by three rapid freeze-thaw cycles. Debris was eliminated by centrifugation at 16 000 g, and the lysates were stored at −80° C. To perform HPLC, a few L of lysate containing about 50 g of protein in about 100 L of 40% acetonitrile and 0.18% TFA was filtered and loaded on a VYDAC C4 column. The globins were then eluted with increasing concentrations of acetonitrile for a period of about 80 minutes. The starting elution buffer was programmed to be 80% buffer A and 20% buffer B and to rise to 50% buffer B in 50 minutes. Buffer A=36% acetonitrile and 0.18% TFA and buffer B=56% acetonitrile and 0.18% TFA. Globin chain elution was monitored by measuring O.D. at 220 nm.

### Example 2

Introduction of the D816V Mutation into iPSCs

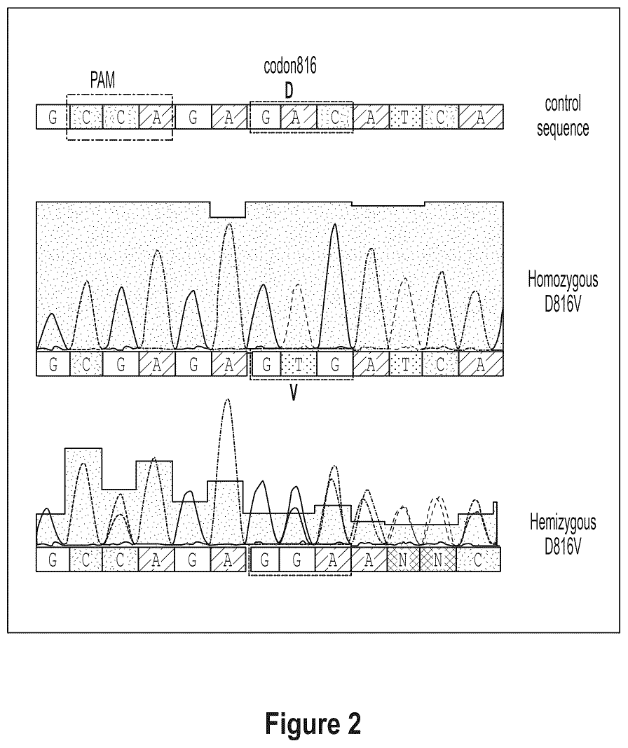

iPSCs from a universal blood donor group O-individual were transfected with Cas9 mRNA, a sgRNA cleaving near position 816 of the SCF receptor, and a 200 bases oligonucleotide coding for the D816V substitution. Screening of about 40 clones yielded 13 homozygous and 6 hemizygous D816V subclones (FIG. 2).

Cas9 mRNA sequence (SEQ ID NO: 3)
ATGGCCCCCAAGAAGAAGCGGAAGGTGGGCATCCACGGCGTGCCCGCCGCCGACAAGAAGTAC
AGCATCGGCCTGGACATCGGCACCAACAGCGTGGGCTGGGCCGTGATCACCGACGAGTACAAG
GTGCCCAGCAAGAAGTTCAAGGTGCTGGGCAACACCGACCGGCACAGCATCAAGAAGAACCTG
ATCGGCGCCCTGCTGTTCGACAGCGGCGAGACCGCCGAGGCCACCCGGCTGAAGCGGACCGCC
CGGCGGCGGTACACCCGGCGGAAGAACCGGATCTGCTACCTGCAGGAGATCTTCAGCAACGAG
ATGGCCAAGGTGGACGACAGCTTCTTCCACCGGCTGGAGGAGAGCTTCCTGGTGGAGGAGGAC
AAGAAGCACGAGCGGCACCCCATCTTCGGCAACATCGTGGACGAGGTGGCCTACCACGAGAAG
TACCCCACCATCTACCACCTGCGGAAGAAGCTGGTGGACAGCACCGACAAGGCCGACCTGCGG
CTGATCTACCTGGCCCTGGCCCACATGATCAAGTTCCGGGGCCACTTCCTGATCGAGGGCGAC
CTGAACCCCGACAACAGCGACGTGGACAAGCTGTTCATCCAGCTGGTGCAGACCTACAACCAG
CTGTTCGAGGAGAACCCCATCAACGCCAGCGGCGTGGACGCCAAGGCCATCCTGAGCGCCCGG
CTGAGCAAGAGCCGGCGGCTGGAGAACCTGATCGCCCAGCTGCCCGGCGAGAAGAAGAACGGC
CTGTTCGGCAACCTGATCGCCCTGAGCCTGGGCCTGACCCCCAACTTCAAGAGCAACTTCGAC
CTGGCCGAGGACGCCAAGCTGCAGCTGAGCAAGGACACCTACGACGACGACCTGGACAACCTG
CTGGCCCAGATCGGCGACCAGTACGCCGACCTGTTCCTGGCCGCCAAGAACCTGAGCGACGCC
ATCCTGCTGAGCGACATCCTGCGGGTGAACACCGAGATCACCAAGGCCCCCCTGAGCGCCAGC
ATGATCAAGCGGTACGACGAGCACCACCAGGACCTGACCCTGCTGAAGGCCCTGGTGCGGCAG
CAGCTGCCCGAGAAGTACAAGGAGATCTTCTTCGACCAGAGCAAGAACGGCTACGCCGGCTAC
ATCGACGGCGGCGCCAGCCAGGAGGAGTTCTACAAGTTCATCAAGCCCATCCTGGAGAAGATG
GACGGCACCGAGGAGCTGCTGGTGAAGCTGAACCGGGAGGACCTGCTGCGGAAGCAGCGGACC
TTCGACAACGGCAGCATCCCCCACCAGATCCACCTGGGCGAGCTGCACGCCATCCTGCGGCGG
CAGGAGGACTTCTACCCCTTCCTGAAGGACAACCGGGAGAAGATCGAGAAGATCCTGACCTTC
CGGATCCCCTACTACGTGGGCCCCCTGGCCCGGGGCAACAGCCGGTTCGCCTGGATGACCCGG
AAGAGCGAGGAGACCATCACCCCCTGGAACTTCGAGGAGGTGGTGGACAAGGGCGCCAGCGCC
CAGAGCTTCATCGAGCGGATGACCAACTTCGACAAGAACCTGCCCAACGAGAAGGTGCTGCCC
AAGCACAGCCTGCTGTACGAGTACTTCACCGTGTACAACGAGCTGACCAAGGTGAAGTACGTG
ACCGAGGGCATGCGGAAGCCCGCCTTCCTGAGCGGCGAGCAGAAGAAGGCCATCGTGGACCTG
CTGTTCAAGACCAACCGGAAGGTGACCGTGAAGCAGCTGAAGGAGGACTACTTCAAGAAGATC
GAGTGCTTCGACAGCGTGGAGATCAGCGGCGTGGAGGACCGGTTCAACGCCAGCCTGGGCACC
TACCACGACCTGCTGAAGATCATCAAGGACAAGGACTTCCTGGACAACGAGGAGAACGAGGAC
ATCCTGGAGGACATCGTGCTGACCCTGACCCTGTTCGAGGACCGGGAGATGATCGAGGAGCGG
CTGAAGACCTACGCCCACCTGTTCGACGACAAGGTGATGAAGCAGCTGAAGCGGCGGCGGTAC
ACCGGCTGGGGCCGGCTGAGCCGGAAGCTGATCAACGGCATCCGGGACAAGCAGAGCGGCAAG
ACCATCCTGGACTTCCTGAAGAGCGACGGCTTCGCCAACCGGAACTTCATGCAGCTGATCCAC
GACGACAGCCTGACCTTCAAGGAGGACATCCAGAAGGCCCAGGTGAGCGGCCAGGGCGACAGC
CTGCACGAGCACATCGCCAACCTGGCCGGCAGCCCCGCCATCAAGAAGGGCATCCTGCAGACC
GTGAAGGTGGTGGACGAGCTGGTGAAGGTGATGGGCCGGCACAAGCCCGAGAACATCGTGATC
GAGATGGCCCGGGAGAACCAGACCACCCAGAAGGGCCAGAAGAACAGCCGGGAGCGGATGAAG
CGGATCGAGGAGGGCATCAAGGAGCTGGGCAGCCAGATCCTGAAGGAGCACCCCGTGGAGAAC
ACCCAGCTGCAGAACGAGAAGCTGTACCTGTACTACCTGCAGAACGGCCGGGACATGTACGTG
GACCAGGAGCTGGACATCAACCGGCTGAGCGACTACGACGTGGACCACATCGTGCCCCAGAGC
TTCCTGAAGGACGACAGCATCGACAACAAGGTGCTGACCCGGAGCGACAAGAACCGGGGCAAG
AGCGACAACGTGCCCAGCGAGGAGGTGGTGAAGAAGATGAAGAACTACTGGCGGCAGCTGCTG
AACGCCAAGCTGATCACCCAGCGGAAGTTCGACAACCTGACCAAGGCCGAGCGGGGCGGCCTG
AGCGAGCTGGACAAGGCCGGCTTCATCAAGCGGCAGCTGGTGGAGACCCGGCAGATCACCAAG
CACGTGGCCCAGATCCTGGACAGCCGGATGAACACCAAGTACGACGAGAACGACAAGCTGATC
CGGGAGGTGAAGGTGATCACCCTGAAGAGCAAGCTGGTGAGCGACTTCCGGAAGGACTTCCAG
TTCTACAAGGTGCGGGAGATCAACAACTACCACCACGCCCACGACGCCTACCTGAACGCCGTG
GTGGGCACCGCCCTGATCAAGAAGTACCCCAAGCTGGAGAGCGAGTTCGTGTACGGCGACTAC
AAGGTGTACGACGTGCGGAAGATGATCGCCAAGAGCGAGCAGGAGATCGGCAAGGCCACCGCC
AAGTACTTCTTCTACAGCAACATCATGAACTTCTTCAAGACCGAGATCACCCTGGCCAACGGC
GAGATCCGGAAGCGGCCCCTGATCGAGACCAACGGCGAGACCGGCGAGATCGTGTGGGACAAG
GGCCGGGACTTCGCCACCGTGCGGAAGGTGCTGAGCATGCCCCAGGTGAACATCGTGAAGAAG
ACCGAGGTGCAGACCGGCGGCTTCAGCAAGGAGAGCATCCTGCCCAAGCGGAACAGCGACAAG
CTGATCGCCCGGAAGAAGGACTGGGACCCCAAGAAGTACGGCGGCTTCGACAGCCCCACCGTG
GCCTACAGCGTGCTGGTGGTGGCCAAGGTGGAGAAGGGCAAGAGCAAGAAGCTGAAGAGCGTG
AAGGAGCTGCTGGGCATCACCATCATGGAGCGGAGCAGCTTCGAGAAGAACCCCATCGACTTC
CTGGAGGCCAAGGGCTACAAGGAGGTGAAGAAGGACCTGATCATCAAGCTGCCCAAGTACAGC
CTGTTCGAGCTGGAGAACGGCCGGAAGCGGATGCTGGCCAGCGCCGGCGAGCTGCAGAAGGGC
AACGAGCTGGCCCTGCCCAGCAAGTACGTGAACTTCCTGTACCTGGCCAGCCACTACGAGAAG
CTGAAGGGCAGCCCCGAGGACAACGAGCAGAAGCAGCTGTTCGTGGAGCAGCACAAGCACTAC
CTGGACGAGATCATCGAGCAGATCAGCGAGTTCAGCAAGCGGGTGATCCTGGCCGACGCCAAC
CTGGACAAGGTGCTGAGCGCCTACAACAAGCACCGGGACAAGCCCATCCGGGAGCAGGCCGAG
AACATCATCCACCTGTTCACCCTGACCAACCTGGGCGCCCCCGCCGCCTTCAAGTACTTCGAC
ACCACCATCGACCGGAAGCGGTACACCAGCACCAAGGAGGTGCTGGACGCCACCCTGATCCAC
CAGAGCATCACCGGCCTGTACGAGACCCGGATCGACCTGAGCCAGCTGGGCGGCGACAGCGGC
GGCAAGCGGCCCGCCGCCACCAAGAAGGCCGGCCAGGCCAAGAAGAAGAAGGGCAGCTACCCC
TACGACGTGCCCGACTACGCCTGA
sgRNA spacer (SEQ ID NO: 4):
AGAATCATTCTTGATGTCTC
Oligo 200 bp for HR (SEQ ID NO: 5):
ACCTAATAGTGTATTCACAGAGACTTGGCAGCCAGAAATATCCTCCTTACTCATGGTCGGATC
ACAAAGATTTGTGATTTTGGTCTAGCGAGAGTGATCAAGAATGATTCTAATTATGTGGTTAAA
GGAAACGTGAGTACCCATTCTCTGCTTGACAGTCCTGCAAAGGATTTTTAGTTTCAACTTTCG
ATAAAAATTGT (Oligos have PAM modified CCA to CGA and D816V GAC
to GTG)

### Differentiation

To assess the effect of the D816V kit mutation, control 01 iPSCs were differentiated, and hemizygous clone A4 and homozygous and B34 were randomly selected using the PSC-RED protocol with or without SCF and Epo (FIG. 3A). Dasatinib, which can be used to inhibit the activity of the D816V SCF receptor, was also included in some experiments. Cells were counted once a week, and differentiation was evaluated by flow cytometry, methyl-cellulose assays, and microscopy after Romanowsky staining. Unedited cells grown in the absence of cytokines were all dead by day 24 and were not further studied.

As shown in FIG. 3B, hemizygous clone A4 grown without SCF and Epo unexpectedly produced over 200,000 mature erythroid cells per iPSCs at day 38, a yield that was almost identical to that of the control cells grown with cytokines. Although the growth curve of the homozygous sub-line was slower during the first 17 days, the culture eventually reached almost the same yield as the other two cultures at day 38.

However, while the control and the hemizygous lines matured and enucleated (see below) when cultured in basal R6 media without dexamethasone between day 38 and day 45, the homozygous cells did not survive this last step and died massively within 5 days, indicating that this sub-line was not able to fully complete the erythroid program.

### Flow Cytometry

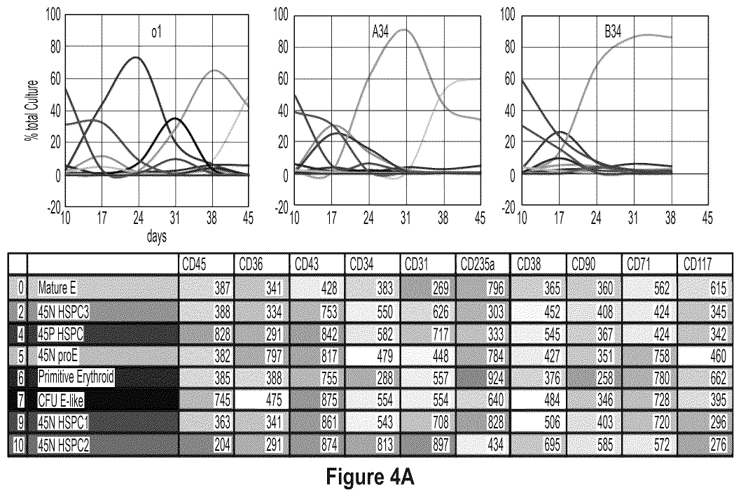

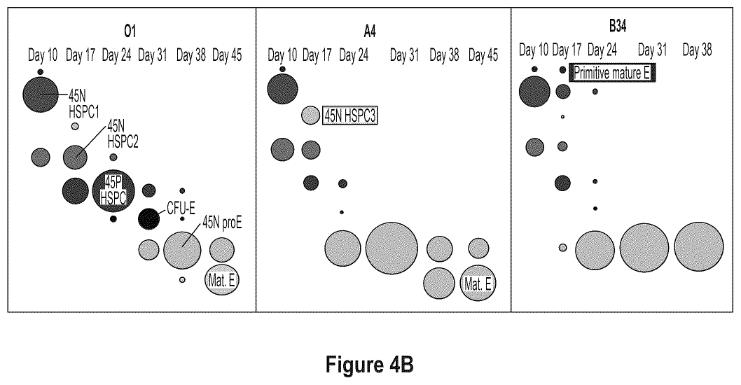

To gain more insights into these cultures, the flow cytometry data were examined. Although fifteen markers were used during the analysis, dimensionality reduction analysis using flowSOM and t-SNE algorithms revealed that the evolution of the cells in culture could be recapitulated by dividing the cells into 8 major populations defined by expression of CD43, CD34, CD31, CD45, CD235a, CD71, and CD117 (FIG. 4A). At day 10, the phenotypes of the cultures of the unmodified and of the two kit mutated lines were similar and composed mostly of populations of CD43+CD45-CD34+ HPSCs, which differed by expression of CD235a (FIG. 4B).

In the control cells, a population of definitive CD45+ HSPCs became predominant at days 17 and 24 and eventually differentiated into CFU-Es and pro-erythroblasts, and finally into mature erythrocytes. In the D816V lines, the CD45+ HSPCs were present but did not amplify to the same extent as the control cells. Instead, a pro-erythroblast population rapidly became pro-eminent. Those cells were able to differentiate into mature erythroblasts in the case of the hemizygous clone, but not in the case of the homozygous clone.

These experiments indicated that it is possible to generate mature erythrocytes from iPSCs without using any SCF and Epo in the culture media but that excessive stimulation by the constitutive SCF receptor led to rapid cell death at a late stage of erythroid differentiation.

The cells obtained at the end of the culture were further examined using HPLC to assess globin expression, and flow cytometry and Romanovsky staining to assess enucleation.

### Enucleation

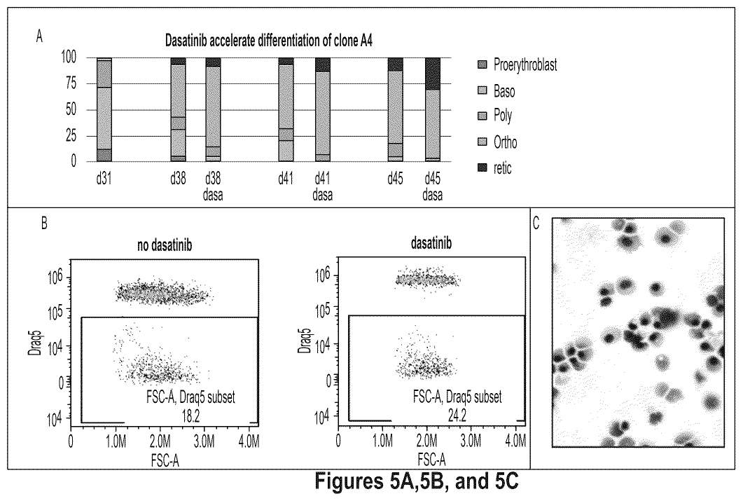

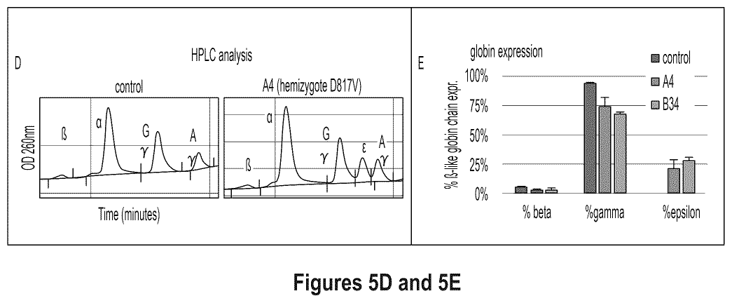

The rate of enucleation was determined by flow cytometry using DRAQ5, a cell permeant DNA dye. Since no cytokines are necessary after day 17 in the PSC-RED protocol in the absence of SCF and Epo, the only trigger left to induce terminal differentiation is the removal of dexamethasone and introduction of RU-4816, a glucocorticoid antagonist (FIG. 2A). To gain more control over terminal differentiation, the effect of dasatanib—an SRC-family protein-tyrosine kinase inhibitor known to be active against the D816V kit mutation—introduced in some cultures starting at day 35 was analyzed. This analysis revealed that the control iPSCs and the A4 clones enucleated at a similar rate of about 18%. Addition of dasatinib on cells from the A4 dramatically accelerated the differentiation (FIG. 5A) and increased the enucleation rate to about 25% (FIGS. 5B and C).

The above results indicated that iPSCs carrying the D816V mutation in the hemizygous state can expand and enucleate in the absence of SCF and Epo at about the same rate as control cells grown in the presence of SCF and Epo. In humans, hemoglobin expression follows a complex pattern. Primitive erythroblasts express mostly embryonic hemoglobins (Hbs), while definitive erythroblasts express first fetal Hb (HbF) and switch to adult Hb (Hb A) after birth. It was previously shown that the erythroblasts obtained using the PSC-RED protocols express mostly fetal globin chains (gamma-globins) and a small amount of adult globin chains (beta-globins). The HPLC analysis of globin expression revealed that the control cells expressed 94% gamma-globin and 6% adult globin (FIGS. 5D-E). By contrast, the D816V hemizygous cells expressed about 75% gamma-globin, 22% epsilon-globin (embryonic), and 3% beta-globin. In accordance with the flow cytometry data, erythroblasts derived from D816V iPSCs are a mixture of primitive cells which cannot enucleate and of definitive cells that can.

D816V iPSCs can be differentiated into enucleated RBCs in the absence of any SCF and Epo using the PSC-RED protocol without any decrease in cell yield. This is an important improvement to the PSC-RED protocol because, in the presence of this mutation, no cytokines are necessary after day 17 of differentiation. The PSC-RED protocol yields about 2,000 cells/iPSCs at day 17 and 100 fold more at day 38 (>200,000/iPSCs). As more than 98% of costs for cell expansion and cell culture occur after day 17, elimination of cytokines after day 17 dramatically decreases the costs of RBC production.

### Example 3

Translational applications of cultured red blood cells differentiated from iPSCs are hampered by high costs, due at least in part to the complexity of the existing differentiation protocols. To simplify the process, various culture protocols have been tested to generate an immortal or semi-immortal cell line that can be grown in a simple chemically-defined media and rapidly differentiated into enucleated cells.

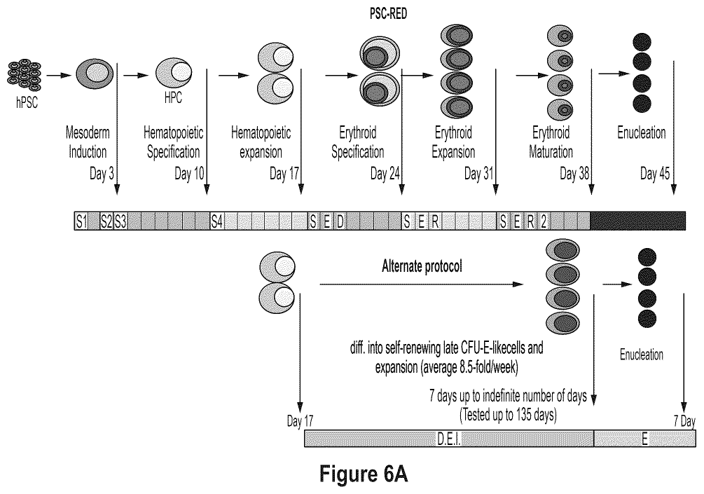

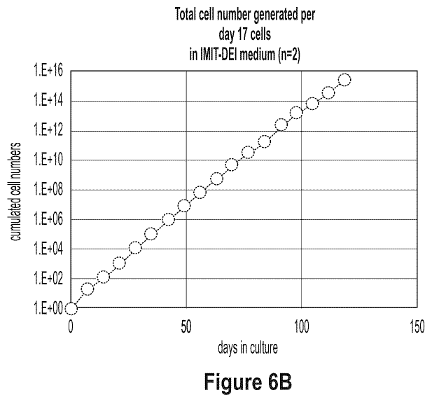

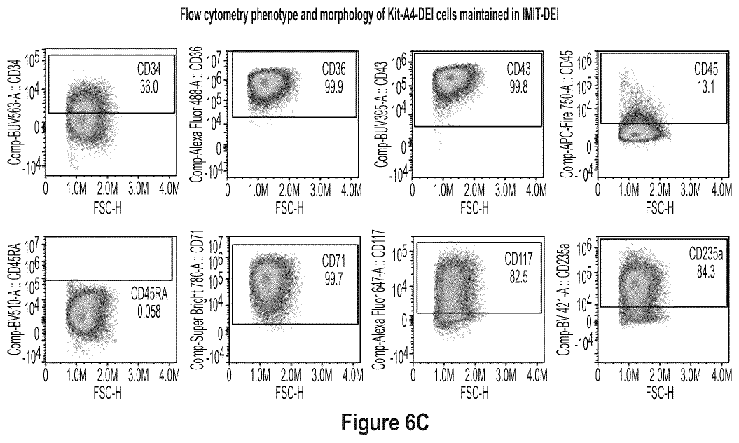

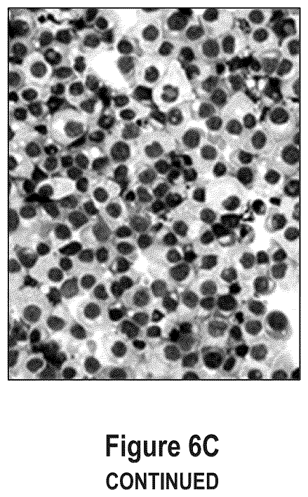

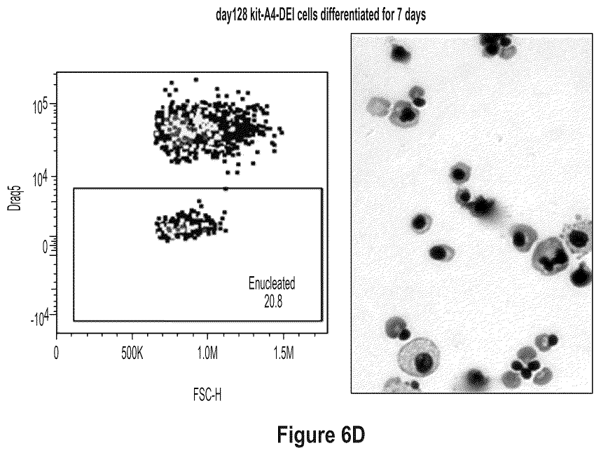

It was demonstrated in this example that cells obtained by differentiating the iPSC A4 line (which contains the kit D816V mutation) for 17 days according to the long PSC-RED protocol and then placing the resulting cells in a culture media termed IMIT-DEI (composed of IMIT, 1 μM dexamethasone, 1 unit of erythropoietin, and 33 μM of isobutylmethylxanthine, 1-Methyl-3-Iso-butyl-xanthine (IBMX)) yielded a homogeneous cell population, termed Kit-A4-DEI (FIG. 6A)

Notably, the resulting kit-A4-DEI cells were able to self-renew in culture (i.e., proliferate while exhibiting no major change in their flow cytometry profile) for an extended period of time by following a protocol including refreshing the media every 2 days by addition of ⅙ volume of 6× concentrated IMIT-DEI media and passaging the cells by 1 to 8 to 1 to 15 dilution every 7 days, keeping the concentration of cells between 200,000 and 2,000,000 cells at all times.

In these conditions the kit-A4-DEI cells expanded 8- to 12-fold every week (average of 8.5 fold) for at least 135 days (FIG. 6B) and showed no signs of slowing down. Weekly characterization of the cells with multiple antibodies on a CYTEK aurora flow cytometer revealed that their surface antigen profile was very stable over time and similar to late CFU-Es/pro-erythroblasts based on the observations that almost all cells expressed CD36, CD43, and CD71, 20) a majority of cells expressed CD117 and CD235a, a minority of cells expressed CD34 and CD45, and almost no cells expressed kit-CD45RA at all time points (FIG. 6D).

Additionally, kit-A4-DEI cells grown in IMIT-DEI can be induced to differentiate at any point by replating them (after centrifugation and at least one wash in PBS) at a concentration of about 300,000 cells/mL in a maturation medium consisting of IMIT and 4 unit of erythropoietin for 7 days. In these conditions, the cells expanded about 4 to 5 times and differentiated into a mixture of cells composed of mature erythroid cells including more than 20% enucleated cells (average 22.3%, n=3, FIG. 6D). The speed of differentiation could be modulated to some degree by adding RU486 (an antagonist of dexamethasone) to eliminate residual traces of dexamethasone in the culture medium.

Other objects, features, and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

1. A method of producing a red blood cell, comprising:
providing a genetically modified pluripotent stem cell comprising a constitutively active stem cell factor (SCF) receptor; and
differentiating the pluripotent stem cell into the red blood cell by culturing the genetically modified pluripotent stem cell in one or more culture media free of a stem cell factor (SCF).

2. The method of claim 1, wherein the SCF receptor comprises a D816V mutation.

3. The method of claim 1, wherein the SCF receptor comprises the amino acid sequence of SEQ ID NO: 2.

4. The method of claim 1, comprising culturing the genetically modified pluripotent stem cell or a derivative thereof in a medium comprising erythropoietin, dexamethasone, and isobutylmethylxanthine, 1-Methyl-3-Iso-butyl-xanthine (IBMX).

5. The method of claim 4, wherein the medium comprises: (a) from about 0.1 to about 10 units of erythropoietin, (b) from about 0.01 μM to about 100 M of dexamethasone, or (c) from about 1 μM to 500 μM of IBMX.

6. (canceled)

7. (canceled)

8. (canceled)

9. (canceled)

10. (canceled)

11. The method of claim 4, wherein the medium comprises about 1 unit of erythropoietin, about 1 μM dexamethasone, about 33 μM of IBMX, or a combination thereof.

12. The method of claim 4, comprising removing the dexamethasone and the IBMX from the medium to induce differentiation of the genetically modified pluripotent stem cell into an enucleated cell.

13. The method of claim 4, comprising refreshing the medium at least once every first interval by adding a concentrated medium comprising erythropoietin, dexamethasone, and IBMX, and passaging cells by dilution at least once every second interval, while maintaining the concentration of the cells between 200,000 and 2,000,000 cells.

14. The method of claim 13, wherein the first interval is about 2 days.

15. The method of claim 13, wherein the second interval is about 7 days.

16. The method of claim 1, wherein the one or more culture media are free of the stem cell factor and erythropoietin, or comprise: (a) a serum-free medium, (b) a defined differentiation medium, (c) a supplement selected from inositol, folic acid, monothioglycerol, transferrin, insulin, ferrous nitrate, ferrous sulfate, BSA, L-glutamine, penicillin-streptomycin, and combinations thereof, or (d) one or more of vascular endothelial growth factor (VEGF), bone morphogenic proteins (BMP), Flt-3L (FL), thrombopoietin (TPO), IL-3, IL-6, and heparin.

17. The method of claim 1, comprising supplementing the one or more culture media with a cytokine only from day 0 to day 17.

18. (canceled)

19. (canceled)

20. (canceled)

21. (canceled)

22. The method of claim 1, further comprising expanding the pluripotent stem cell prior to being differentiated into the red blood cell.

23. The method of claim 1, wherein the pluripotent stem cell is cultured using a bioreactor.

24. The method of claim 1, further comprising differentiating the pluripotent stem cell into a progenitor cell and sorting a population of progenitor cells derived from the pluripotent stem cell using magnetic-activated cell sorting (MACS), flow cytometry, or fluorescence-activated cell sorting (FACS).

25. The method of claim 23, further comprising sorting a population of progenitor cells based on expression of one or more of CD31, CD34, CD43, and CD45.

26. The method of claim 1, further comprising dispersing a population of pluripotent stem cells by treatment with one or more enzymes.

27. The method of claim 25, wherein the one or more enzymes comprise trypsin or TrypLE.

28. The method of claim 1, wherein the red blood cell expresses ADAMTS13, asparaginase, Factor VIII, Factor IX, or phenylalanine hydroxylase.

29. The method of claim 1, wherein the pluripotent stem cell comprises: (a) an embryonic stem cell or an embryo-derived cell, or (b) an induced pluripotent stem cell (iPSC).

30. (canceled)

31. The method of claim 1, wherein the pluripotent stem cell is a human cell.

32. (canceled)

33. (canceled)

34. (canceled)

35. (canceled)

36. (canceled)

37. (canceled)


SEQUENCE LISTING (8 sequences)

SEQ ID NO: 1 - protein, 976 aa, Homo sapiens
RGARGAWDFLCVLLLLLRVQTGSSQPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKGNNKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 2 - protein, 976 aa, synthetic construct
  REGION 1..976: note = Synthetic
RGARGAWDFLCVLLLLLRVQTGSSQPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKGNNKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARVIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 3 - DNA, 4245 bp, synthetic construct
  misc_feature 1..4245: note = Synthetic
  source 1..4245: mol_type = other DNA
atggcccccaagaagaagcggaaggtgggcatccacggcgtgcccgccgccgacaagaagtacagcatcggcctggacatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaagagcgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgagccggaagctgatcaacggcatccgggacaagcagagcggcaagaccatcctggacttcctgaagagcgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggaccacatcgtgccccagagcttcctgaaggacgacagcatcgacaacaaggtgctgacccggagcgacaagaaccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaagagcaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaagaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacagcggcggcaagcggcccgccgccaccaagaaggccggccaggccaagaagaagaagggcagctacccctacgacgtgcccgactacgcctga

SEQ ID NO: 4 - DNA, 20 bp, synthetic construct
  misc_feature 1..20: note = Synthetic
  source 1..20: mol_type = other DNA
agaatcattcttgatgtctc

SEQ ID NO: 5 - DNA, 200 bp, synthetic construct
  misc_feature 1..200: note = Synthetic
  source 1..200: mol_type = other DNA
acctaatagtgtattcacagagacttggcagccagaaatatcctccttactcatggtcggatcacaaagatttgtgattttggtctagcgagagtgatcaagaatgattctaattatgtggttaaaggaaacgtgagtacccattctctgcttgacagtcctgcaaaggatttttagtttcaactttcgataaaaattgt

SEQ ID NO: 6 - DNA, 13 bp, synthetic construct
  source 1..13: mol_type = other DNA
gccagagacatca

SEQ ID NO: 7 - DNA, 13 bp, synthetic construct
  source 1..13: mol_type = other DNA
gcgagagtgatca

SEQ ID NO: 8 - DNA, 13 bp, synthetic construct
  source 1..13: mol_type = other DNA
gccagaggaatca